(19) United States
(12) Patent Application Publication
(10) Pub. No.: US 2026/0345321 A1
(43) Pub. Date: Nov. 5, 2026

(54) PROSTHETIC VALVE LEAFLET ATTACHMENT

(71) Applicant: Edwards Lifesciences Corporation, Irvine, CA (US)

(72) Inventors: Hannah Reed Bettencourt, Huntington Beach, CA (US); Ngoc Huong Thi Nguyen, Costa Mesa, CA (US)

(21) Appl. No.: 19/713,038

(22) Filed: Jun. 18, 2026

Related U.S. Application Data
(63) Continuation of application No. PCT/US2024/058936, filed on Dec. 6, 2024.
(60) Provisional application No. 63/613,408, filed on Dec. 21, 2023.

(51) Int. Cl. A61F 2/24

(52) U.S. Cl. CPC A61F 2/2418; A61F 2/2415; A61F 2220/0075

(57) ABSTRACT

A prosthetic heart valve includes an annular frame, a skirt attached to the frame, and a leaflet structure disposed within the frame and coupled to a commissure portion of the frame. The leaflet structure includes a first leaflet comprising a first lateral edge and a second leaflet comprising a second lateral edge coupled to the first lateral edge of the first leaflet in a leaflet-to-leaflet union such that the first lateral edge and the second lateral edge are pointed radially inward with respect to an axis of the annular frame.

### RELATED APPLICATION(S)

This application is a continuation of PCT Patent Application No. PCT/US 2024/058936 filed on Dec. 6, 2024, which claims the benefit of U.S. Provisional Patent Application No. 63/613,408, filed on Dec. 21, 2023, the complete disclosure of each of these applications being hereby incorporated by reference in its entirety.

### BACKGROUND

Certain prosthetic heart valves include valvular leaflet structures that comprise two or more leaflets, or leaflet portions, that are attached to one another, or to other structure of the valve, in one or more areas/portions. The attachment configuration of such leaflets can affect the motion of the leaflets under certain pressure conditions.

### SUMMARY

Described herein are devices, methods, and systems relating to prosthetic heart valves. Such prosthetic valves can include leaflet attachment features that promote leaflet opening and/or other leaflet motion under certain pressure conditions. Prosthetic valves of the present disclosure can include leaflets attached together and/or to a frame or other structure of the valve in commissure area(s) of the valve. Whereas some leaflet attachment designs join/couple adjacent leaflet edges in a configuration with the leaflet edges oriented radially outwardly relative to the valve axis, examples of the present disclosure include leaflet unions that hold adjacent leaflet edges together such that the edges are oriented radially inwardly. Such leaflet unions can be secured/fixed using a comb/blanket stitch, or other attachment means, such as adhesive, or other type(s) of stitches. Leaflet unions/junctions of examples of the present disclosure can be implemented/disposed in an area near and/or below commissure tab features of the joined/attached leaflets (with respect to a vertical orientation as illustrated in various figures of the present disclosure).

Compared to designs in which joined leaflet edges are oriented radially-outwardly, which can cause leaflet portions adjacent to the joined leaflet edges to be inclined/deflected towards the valve axis, in examples in which joined leaflet edges are oriented radially-inwardly, the edge-adjacent leaflet portions can be inclined away from the valve axis, thereby biasing commissure areas of the leaflet assembly (e.g., the area(s) of the leaflet union(s)) to have a more-open default/biased configuration.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 shows a perspective view of a prosthetic heart valve in accordance with one or more examples.

FIG. 2 shows a perspective view of a prosthetic heart valve in accordance with one or more examples.

FIG. 3 shows a prosthetic heart valve in a compressed delivery configuration and coupled to a delivery system in accordance with one or more examples.

FIG. 4 shows a prosthetic heart valve deployed in an aortic position of a human heart in accordance with one or more examples.

FIG. 5 shows a perspective view of a prosthetic heart valve in accordance with one or more examples.

FIG. 6 shows a prosthetic heart valve deployed in a docking station in a mitral position of a human heart in accordance with one or more examples.

FIG. 7 shows a perspective view from an outflow end of an annular frame of a prosthetic heart valve in accordance with one or more examples.

FIG. 8A shows a main skirt disposed on a frame of a prosthetic heart valve in accordance with one or more examples.

FIG. 8B shows the main skirt of FIG. 8A in a flattened/plan view in accordance with one or more examples.

FIG. 8C shows an outer skirt disposed on a frame of a prosthetic heart valve in accordance with one or more examples.

FIG. 9 shows a leaflet assembly/structure of a prosthetic heart valve in accordance with one or more examples.

FIG. 10 is a simplified plan view of a single leaflet of a leaflet assembly/structure of a prosthetic heart valve in accordance with one or more examples.

FIG. 11 is a simplified plan view of a pair of coupled leaflets of a valvular leaflet assembly/structure of a prosthetic heart valve in accordance with one or more examples.

FIG. 12 shows leaflets of a valvular leaflet assembly/structure of a prosthetic heart valve coupled at tab portions in accordance with one or more examples.

FIG. 13 shows a leaflet of a valvular leaflet assembly/structure having a reinforcing strip and sub-commissure suturing in accordance with one or more examples.

FIG. 14 shows a simplified cross-sectional view of a commissure portion of a leaflet assembly/structure attached to an annular frame of a prosthetic heart valve in accordance with one or more examples.

FIG. 15 is a close-up perspective view of a commissure portion of a valvular leaflet assembly/structure attached to an annular frame of a prosthetic heart valve in accordance with one or more examples.

FIG. 16A shows a prosthetic heart valve having a leaflet assembly/structure with leaflets including edge-to-edge leaflet unions in accordance with one or more examples.

FIG. 16B shows a sub-commissure portion of adjacent leaflets of the leaflet assembly/structure of the prosthetic heart valve of FIG. 16A with leaflet edge portions joined in a radially-outward orientation in accordance with one or more examples.

FIG. 16C shows an axial view from an outflow end of the prosthetic heart valve of FIGS. 16A and 16B in an open configuration in accordance with one or more examples.

FIG. 17A shows a prosthetic heart valve having a valvular leaflet assembly/structure with leaflets including edge-to-edge leaflet unions in accordance with one or more examples.

FIG. 17B shows an outer view of a sub-commissure portion of adjacent leaflets of the leaflet structure of the prosthetic heart valve of FIG. 17A with leaflet edge portions joined in a radially-inward orientation in accordance with one or more examples.

FIG. 17C shows an inner view of a sub-commissure portion of the leaflet assembly/structure shown in FIG. 17B in accordance with one or more examples.

FIG. 17D shows an axial view from an outflow end of the prosthetic heart valve of FIG. 17A-C in an open configuration in accordance with one or more examples.

FIG. 17E shows an axial view from an inflow end of the prosthetic heart valve of FIG. 17A-D in an open configuration in accordance with one or more examples.

FIG. 18 shows adjacent leaflets of a valvular leaflet assembly/structure coupled at respective commissure portions in accordance with one or more examples.

FIG. 19 shows an inner view of leaflets of a leaflet assembly joined at respective sub-commissure portions in a radially-inward/inverted configuration in accordance with one or more examples.

FIG. 20 shows an outer view of leaflets of a leaflet assembly joined at respective sub-commissure portions in a radially-inward/inverted configuration in accordance with one or more examples.

FIG. 21 shows a leaflet assembly/structure having adjacent leaflets joined with radially-inward/inverted unions in accordance with one or more examples.

FIG. 22 shows an inner-diameter view leaflet assembly/structure coupled to a frame, the leaflet assembly/structure including radially-inward/inverted sub-commissure unions in accordance with one or more examples.

FIG. 23 shows an inner-diameter view of an assembled prosthetic heart valve having a valvular structure for regulating blood flow in a low-pressure gradient hemodynamic situation in accordance with one or more examples.

FIG. 24 shows an assembled prosthetic heart valve having a leaflet assembly/structure for regulating blood flow in a low-pressure gradient hemodynamic situation in accordance with one or more examples.

FIG. 25 is a close-up perspective view of a commissure portion of a leaflet/valvular structure attached including radially-inward/inverted sub-commissure unions in accordance with one or more examples.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to only the numeric portion (e.g., ‘10’) may refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present written description to a feature ‘10’ may be understood to refer to either an identified feature ‘10a’ in a particular figure of the present disclosure or to an identifier ‘10’ or ‘10b’ in the same figure or another figure, as an example. Furthermore, where a reference identifier is used that comprises a first numeric portion followed by a dash (e.g., ‘-’) and a second numeric portion (e.g., ‘10-2,’ where ‘10’ is the first numeric portion and ‘2’ is the second numeric portion), references in the written description to only the first numeric portion (e.g., ‘10’) may refer to any feature identified in the figures using such first numeric portion (e.g., ‘10-1,’ ‘10-2,’ ‘10-3,’ etc.), even where such features are identified with reference identifiers that concatenate the numeric portion thereof with a dash and a second numeric portion. That is, a reference in the present written description to a feature ‘10’ may be understood to refer to either an identified feature ‘10-1’ in a particular figure of the present disclosure or to an identifier ‘10’ or ‘10-2’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

### Transcatheter Heart Valves

Solutions of the present disclosure relate to prosthetic heart valve devices having leaflet couplings and/or configurations that promote desirable leaflet opening behavior in response to certain pressure conditions/gradients. Example valve devices of the present disclosure may advantageously be configured to promote leaflet opening in a manner that produces desirable blood wash-out and leaflet cycling in relatively low pressure conditions. Example prosthetic valves are described below in the context of mitral and aortic valve replacement devices, though it should be understood that valves disclosed herein can be implemented in any cardiac (e.g., pulmonary, tricuspid) or non-cardiac position, or in non-implant applications. The following includes description of human cardiac anatomy that is relevant to certain inventive features and examples disclosed herein.

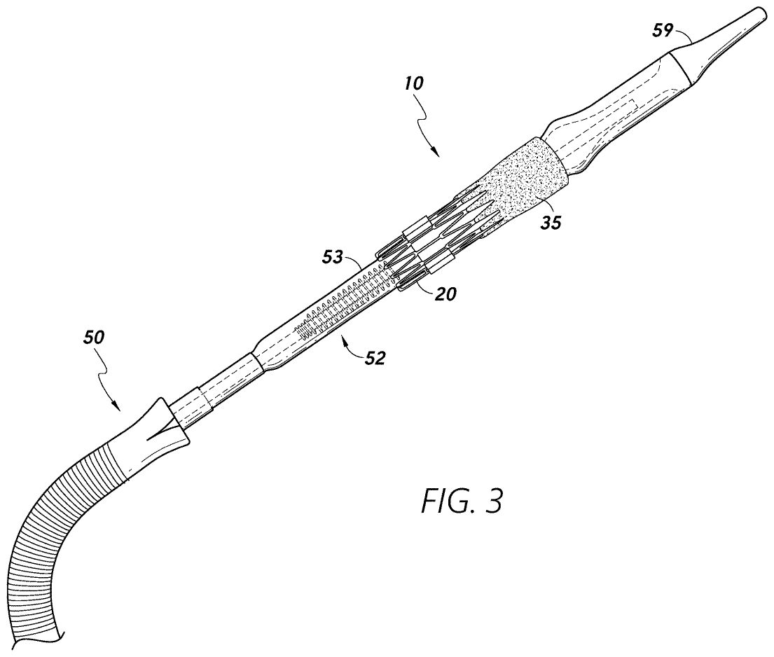

The human heart can suffer from various valvular diseases, which can result in significant malfunctioning of the heart and ultimately require repair of the native valve or replacement of the native valve with an artificial valve. Various repair devices (e.g., stents) and artificial valves can be implemented in repair/replacement treatments. Percutaneous and minimally-invasive surgical approaches are used in various procedures to deliver prosthetic medical devices to locations inside the body that are not readily accessible by surgery or where access without surgery is desirable. For example, a prosthetic heart valve can be mounted in a crimped configuration on/within a distal portion of a delivery device/system and advanced through the patient's vasculature until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery device so that the prosthetic valve can self-expand to its functional size. An expandable transcatheter heart valve disposed in an example delivery system is shown in FIG. 3.

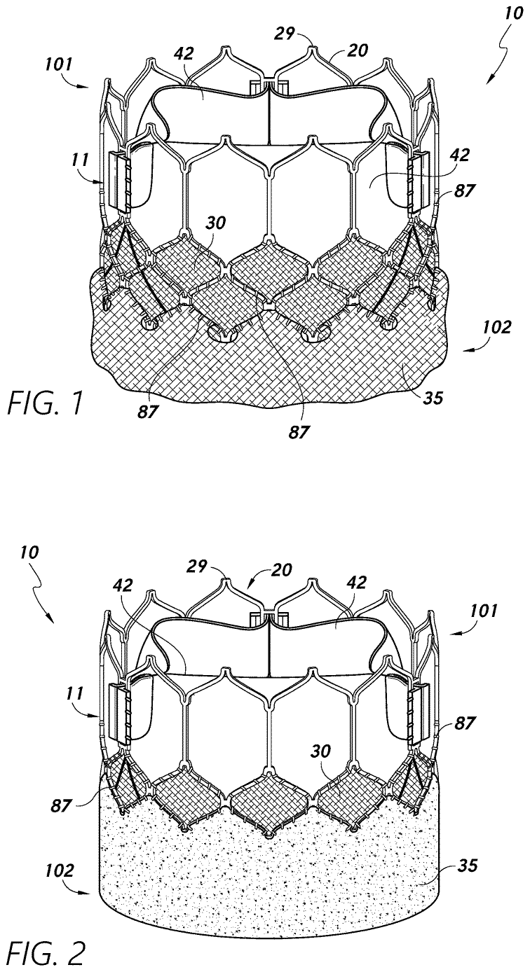

FIG. 1 shows a perspective view of a transcatheter prosthetic heart valve 10 that may be deployed a position within a heart in accordance with one or more examples. The transcatheter heart valve 10 can include an annular metal frame 20, a valvular/leaflet assembly/structure 40 having a plurality of leaflets 42 supported within the frame 20, a main skirt 30 coupled to an interior of the metal frame 20, and an outer skirt 35 coupled to an exterior of the metal frame 20. The valve 10 may have a valve profile similar to the Edwards Lifesciences SAPIEN 3™ valve, for example.

The valvular structure 40 can comprise three leaflets 42, or other number. The valvular structure 40 is coupled to the annular frame 20 near an outflow end 101 of the prosthetic valve 10 by a plurality of commissures 11 formed, for example, by tabs of adjacent leaflets 42. The main skirt 30 can be configured to cover an inner circumferential diameter/surface of the annular frame 20 near the inflow end 102 of the prosthetic valve 10. The outer skirt 35 can be configured to cover an outer circumferential diameter/surface of the frame 20 near the inflow end 102. Each leaflet 42 can have a cusp edge that is attached to the main skirt 30 and/or frame 20. The main skirt 30 and/or the outer skirt 35 can be constructed of a porous material, such as woven, braided or knitted fabrics formed from synthetic fibers, such as polyethylene terephthalate (PET) fibers. The porous nature of the skirt material can encourage cellular ingrowth of the surrounding native tissue while otherwise being substantially impermeable to blood cells in the blood flow through the heart valve. This native tissue ingrowth into the skirts 30, 35 can further secure the heart valve 10 within the native anatomy of the patient and reduce paravalvular leakage (PVL).

FIG. 2 shows a perspective view of a prosthetic heart valve 10 having an alternative outer skirt configuration compared to the implementation of FIG. 1. In particular the configuration of the outer sealing skirt 35 in FIG. 2 is taller than the corresponding feature in the implementation of FIG. 1. The valve 10 implementation shown in FIG. 2 may have a valve profile similar to the Edwards Lifesciences SAPIEN 3 Ultra™ valve, for example.

FIG. 3 shows a prosthetic heart valve 10 as shown in FIG. 1 or FIG. 2 in a compressed delivery configuration and for transport via a delivery system 50 in accordance with one or more examples. A delivery system/apparatus 50 as shown in FIG. 3 can be used in combination with any of the prosthetic heart valves described herein.

FIG. 3 shows the valve 10 crimped on a balloon catheter 52. The geometry of the struts and junctions of the frame 20 can assist in creating enough space in certain of the cell openings thereof in the crimped configuration to allow portions of the valve leaflets 42 to protrude/bulge outwardly through the frame cell openings. Such design can allow the valve 10 to be crimped to a relatively small diameter compared to implementations in which all of the leaflet material is constrained within the crimped frame.

The delivery system 50 can includes a handle (not shown) that can be disposed external to a patient and used to articulate a distal end portion of an elongated shaft of the delivery system 50 within a patient. The prosthetic heart valve 10 can be disposed on the distal end portion 53 of the balloon catheter 52 in a radially-compressed state. For example, the prosthetic valve 10 can be crimped on the inflatable balloon 52 or another type of expansion member that can be used to radially expand the prosthetic valve 10 when deployed. The distal end portion 53, including the prosthetic valve 10, can be advanced through the patient's vasculature to a selected implantation site (e.g., within a native mitral valve). Although not specifically illustrated in FIG. 3, the delivery system 50 can be advanced over a guidewire, wherein the delivery apparatus 50 can include an innermost shaft that defines a lumen for the guidewire. The prosthetic valve 10 can be deployed at the implantation site, such as by inflating the balloon 52. Further details of delivery apparatuses that can be used to deliver and implant plastically-expandable prosthetic heart valves, such as the prosthetic valve 10 (or any other of the prosthetic heart valves disclosed herein), are disclosed in U.S. Patent Application Publication Nos. 2017/0065415, 2016/0158497, and 2013/0030519, which are incorporated herein by reference.

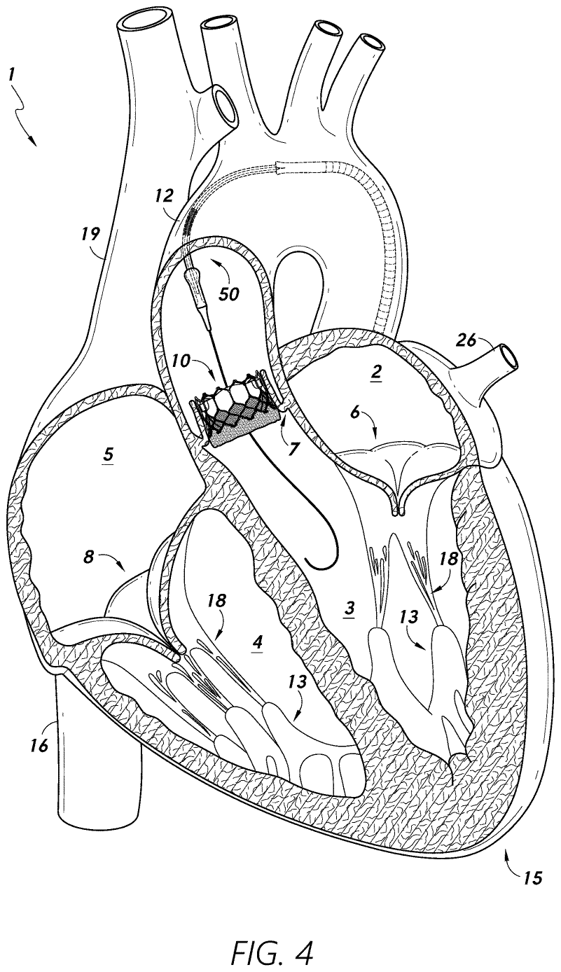

FIG. 4 shows a prosthetic heart valve 10 as shown in any of FIGS. 1-3 deployed in an aortic position of a human heart 1 in accordance with one or more examples. The valve 10 may be implanted in a position within leaflets of the native aortic valve 7.

Referring generally to the cardiac anatomy shown in FIG. 4, in humans and other vertebrate animals, the heart 1 generally comprises a muscular organ having four pumping chambers, wherein the flow of blood between the pumping chambers is at least partially controlled by various heart valves, namely, the aortic 7, mitral (or bicuspid) 6, tricuspid 8, and pulmonary (not shown for visual clarity) valves. The valves may be configured to open and close in response to a pressure gradient present during various stages of the cardiac cycle (e.g., relaxation and contraction) to at least partially control the flow of blood to a respective region of the heart and/or to associated blood vessels (e.g., pulmonary, aorta, etc.).

The left ventricle 3 is the primary pumping chamber of the heart 1. A healthy left ventricle is generally conical or apical in shape and descends from a base with a decreasing cross-sectional diameter and/or circumference to the point or apex 15. The pumping of blood from the left ventricle 3 is accomplished by a squeezing motion and a twisting or torsional motion resulting from contraction of heart muscle fibers that extend in a circular or spiral direction around the heart.

The valves of the heart 1 aid the circulation of blood in the heart. The tricuspid valve 8 separates the right atrium 5 from the right ventricle 4. The tricuspid valve 8 generally has three cusps or leaflets and advantageously closes during ventricular contraction (i.e., systole) and opens during ventricular expansion (i.e., diastole). The pulmonary valve separates the right ventricle 4 from the pulmonary artery (not shown) and generally is configured to open during systole so that blood may be pumped toward the lungs from the right ventricle 4, and close during diastole to prevent blood from leaking back into the right ventricle 4 from the pulmonary artery. The pulmonary valve generally has three cusps/leaflets.

The mitral valve 6 generally has two cusps/leaflets and separates the left atrium 2 from the left ventricle 3. The mitral valve 6 may generally be configured to open during diastole so that blood in the left atrium 2 can flow into the left ventricle 3, and close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The atrioventricular (i.e., mitral 6 and tricuspid 8) heart valves are generally associated with a sub-valvular structure, including a collection of chordae tendineae 18 and papillary muscles 13 securing the leaflets of the respective valves to promote and/or facilitate proper coaptation of the valve leaflets and prevent prolapse thereof. The papillary muscles 13, for example, may generally comprise finger-like projections from the ventricle wall.

The primary roles of the left atrium 2 are to act as a holding chamber for blood returning from the lungs (not shown) and to act as a pump to transport blood to other areas of the heart. The left atrium 2 receives oxygenated blood from the lungs via the pulmonary veins 26. The oxygenated blood that is collected from the pulmonary veins 26 in the left atrium 2 enters the left ventricle 3 through the mitral valve 6. Deoxygenated blood enters the right atrium 5 through the inferior 16 and superior 19 venae cavae. The right side of the heart then pumps this deoxygenated blood into the pulmonary arteries around the lungs. There, fresh oxygen enters the blood stream, and the blood moves to the left side of the heart via a network of pulmonary veins ultimately terminating at the left atrium 2, as shown. Any of the above noted native heart valves may fail to operate properly, for example, by allowing blood to backflow therethrough or regurgitate into an upstream heart chamber or blood vessel.

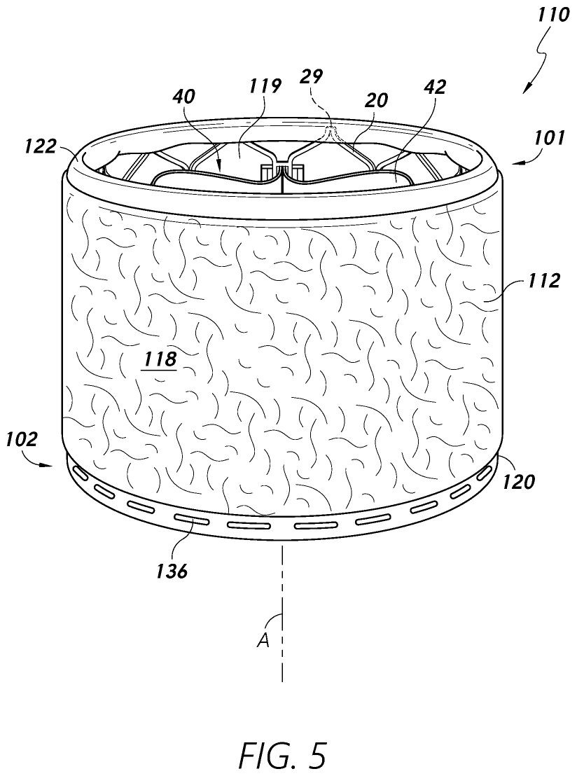

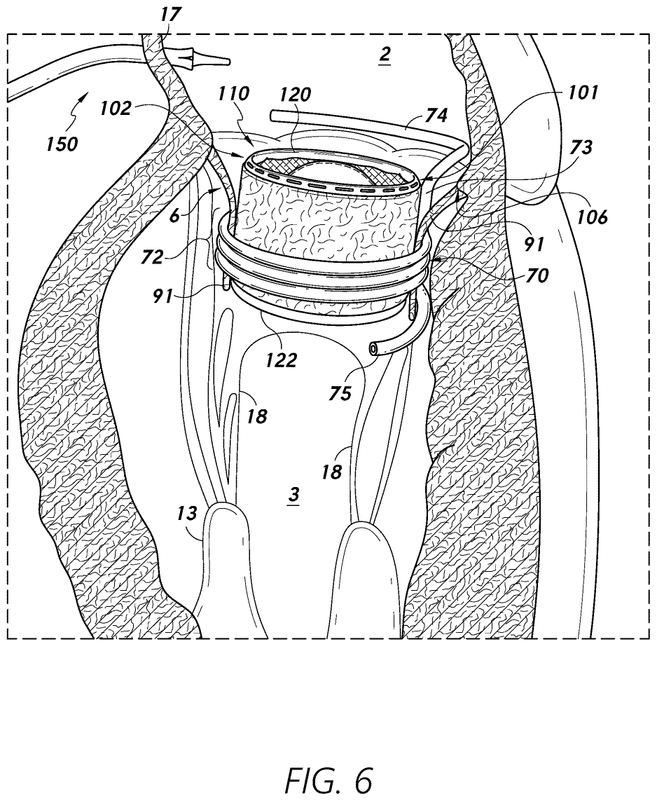

FIG. 5 shows a perspective view of a prosthetic heart valve 110 in accordance with one or more examples. FIG. 6 shows the prosthetic heart valve 110 deployed in a helical docking device 70 in a mitral position of a human heart in accordance with one or more examples. The prosthetic valve 110 may have any of the features of any of the heart valves described above in connection with FIG. 1-4. Although the valve 110 is shown as implanted in a mitral position within the mitral valve annulus, it should be understood that the valve 110 may be implanted in a similar manner in a position within the tricuspid valve. The description below can be understood relative to both FIGS. 5 and 6.

As shown, the left side of the heart includes the left atrium 2, the left ventricle 3, and the mitral valve 6 connecting the left atrium 2 and the left ventricle 3. The mitral valve 6 includes anterior and posterior leaflets 91 that are connected to an inner wall of the left ventricle 3 via chordae tendineae 18 and papillary muscles 13. The prosthetic valve 110 may be implanted within the mitral valve annulus 106 via the docking device 70. The docking device 70 can include one or more central turns/coils 72 that are configured to be placed around the native leaflets 91 of the native valve 6 and/or the chordae tendineae 18. The coils 72 can advantageously pinch/urge portions of the leaflets 91 inwards in order to form a more circular opening at the native valve for more effective implantation of the prosthetic valve 110. The valve prosthesis 110 can be retained at the native valve 6 by the docking device 70. When delivered (e.g., using the delivery system 150) and expanded within the coils 72 of the docking device 70, the prosthetic valve 110 pushes outwardly against the coils 72 to secure the positions of both the valve 110 and the docking device 70. In some examples, an undersized docking device 70 with an inner diameter that is slightly smaller than the diameter of the prosthetic valve 110 in its expanded state can be used to provide a more secure fitting between the parts. As can be seen in FIG. 6, at least a portion of the native valve leaflets 91 and/or a portion of the chordae tendineae 18 can be pinched or sandwiched between the valve 110 and the docking device 70 to secure the components to the native anatomy.

The helical docking device/anchor 70 can extend both upstream and downstream of the native valve 6. In some implementations, the docking device 70 can provide a greater and more secure attachment area against which the prosthetic valve 110 can abut compared to implantation within a native valve annulus without such a docking device. Methods and devices for implanting anchors/docking devices and prosthetic valves, which can be used with the inventions in this disclosure, are described in U.S. Pub. No. 2018/0055628, U.S. Pub. No. 2018/0055630, U.S. Pub. No. 2018/0177594, and U.S. Pub. No. 2018/0318079, which are each incorporated herein by reference.

The docking device/anchor 70 includes a functional coil/turn portion/region 72, which may be positioned centrally with respect to the axis of the device 70. The docking device 70 can further include an encircling arm/portion 75, which may be positioned below the functional coil portion 72 (with respect to the vertical orientation shown in FIG. 6). The docking device 70 can further include an upper atrial stabilizing arm/portion 74.

The anatomy-encircling arm 75 can include an elongate curved extension forming an arc and/or one or more turns that can be configured to encircle or capture the chordae tendineae 18 and/or the leaflets 91 of the native valve 6; although shown as a mitral valve 6, it should be understood that the valve 110 and docking device 70 may be deployed in a tricuspid valve. The central functional portion 72 can include a plurality of turns/coils configured to retain the prosthetic valve 110 at the native valve 6.

The atrial arm 74 can include an elongate curved extension forming an arc and/or one or more turns configured to keep the docking device 70 from being dislodged from the valve annulus prior to implantation of the prosthetic valve 110. In some implementations, the upper arm 74 can be positioned over the floor of the atrium 2, and can be configured to keep the turns of the central region 72 positioned high within the native valve apparatus.

The docking device 70 can be introduced to the target chamber(s) using a delivery system 150, with may be similar to other delivery systems/catheters disclosed herein. When advancing the distal portion of the delivery system 150 to the target implantation site, the delivery system 150 may hold the docking device 70 in a low-profile delivery configuration. For example, in the delivery configuration, the coil form of the docking device 70 may be elongated/straightened, such that the axis of the elongate tube/form of the device 70 is substantially parallel with the axis of the delivery system 150. In some implementations, the delivery system may be advanced to the target site via a transseptal approach, as shown in FIG. 6. When deployed from the delivery system 150, the elongate tube/shaft of the docking device 70 may then be coiled/wound around the native mitral leaflets 91 within the ventricle 3, thereby forming a cupholder-type shape that serves as the anchor for the valve device 110. The atrial arm/turn 74, which may or may not be sutured to the atrial anatomy, can maintain the dock position prior to valve deployment.

The docking device 70 can include a vertical extension portion 73 between the central coil(s) 72 and the upper atrial arm 74. The extension portion 73 can include a part of the coil that extends substantially parallel to a central axis of the anchor coils 72. The extension portion/connector 73 can serve to vertically offset/space (e.g., in direction of axis of central portion 72) the arm 74 from the coils 72, which may form a gap between the atrial side and the ventricular side of the docking device 70. The extension portion 73 can be configured to be positioned through, near, and/or around the native valve annulus 106, in order to reduce the amount of the device 70 that passes through, pushes, or rests against the native annulus and/or the native leaflets 91 when the anchor 70 is implanted. This can reduce the force applied by the device 70 on the native valve and reduce abrasion of the native leaflets. In some implementations, the extension portion 73 is positioned at and/or passes through one of the commissures of the native valve, thereby spacing the upper arm 74 from the native leaflets of the native valve to prevent the atrial arm 74 from interacting with the native leaflets from the atrial side. The extension portion 73 can also serve to elevate the upper arm 74 such that it contacts the atrial wall above the native valve, which can reduce the stress on and around the native valve 6, as well as provide for better retention of the anchor.

The docking device can further include one or more openings or other attachment means (e.g., through holes) at one or more ends of the anchor tube/shaft 70, which may facilitate attaching a cover layer over the tube/shaft of the anchor/device 70 and/or may provide an attachment site or tethering holes for delivery tools such as a pull wires, retention members, retention sutures, etc. In some examples, a width or thickness of the tube/shaft of the docking device 70 can also be varied along the length thereof. For example, the central portion 72 of the device 70 and/or extension 73 can be made thinner than end portions of the device. This can allow the central portion 72 and/or extension 73 to exhibit greater flexibility, while the end portions can be stronger or more robust. In certain examples, making the end portions of the device/coil relatively thicker can also provide more surface area for suturing or otherwise attaching a cover layer to the coil of the device/anchor 70. The docking device 70 can be formed at least in part of nitinol or other shape-memory material.

The docking device 70 can be configured for insertion through the native valve annulus in a counter-clockwise (or clockwise) direction. For example, the docking device 70 can be advanced through commissure A3P3, commissure A1P1, or through another part of the native mitral valve. The counter-clockwise direction/chirality of the coil of the device 70 can also allow for bending of the distal end of the delivery catheter 150 in a similar counter-clockwise direction, which can be easier to achieve than to bend the delivery catheter in the clockwise direction. However, it should be understood that the anchor can be configured for either clockwise or counter-clockwise insertion through the valve, as desired. The docking device 70, in view of the various features described above, can provide various advantages with respect to atrioventricular in-valve docking, including: low-profile; transeptal delivery and retrievability; treatment of a wide range of annulus sizes; atrial turn(s) that maintains dock position prior to valve deployment; functional turn(s) that provide anchor for valve; and/or encircling ventricular arm/turn(s) that guide implant in capturing native leaflets.

Once the docking device 70 is delivered within the native valve annulus, which may be accomplished with a dedicated delivery system, the prosthetic valve 110 may be introduced separately and expanded within the dock 70. The illustrated delivery system 150 may represent the delivery system for the docking device 70, the prosthetic valve 110, or a common delivery system that may be used for delivery of both the dock 70 and the valve 110.

In some implementations, where the valve implant 110 is deployed within a docking device like the docking device 70 shown, the prosthetic valve 110 may advantageously be a skirted transcatheter valve including an outer skirt 112 to improve secure coupling of the valve 110 and/or reduce the risk of damage to adjacent anatomical tissue and/or device component(s). For example, the valve 110 may be similar to any of the valve devices described above in connection with FIG. 1-4, but may further include the additional outer skirt 112. As described above, the prosthetic valve 110 can include a metal frame 20 that forms a number of edged crowns or apices 29 and protruding commissure attachment posts 11, as well as suture(s) 87 that can be exposed along an outer surface of the frame 20. These features can cause damage to the native tissue, such as tissue lodged between the prosthetic valve 110 and the docking device/anchor 70, for example, by movement or friction between the native tissue and the various abrasive surfaces of the prosthetic valve 110. In addition, other native tissue in close proximity to the prosthetic valve 110, such as the chordae tendineae, can also potentially be damaged in the absence of the outer skirt 112.

The skirt 112 may comprise knitted polyethylene terephthalate (PET), or other material, and may serve to aid in sealing between the valve 110, the native leaflets 91, and/or the dock/anchor 70. In the deployed position shown in FIG. 6, the docking device 70 advantageously can encircle the native mitral leaflets 91 to secure the valve system 110.

Aside from the skirt 112, the valve 110 may be similar to any of the prosthetic valves 10 described above. For example, the valve 110 may include a frame 20, a leaflet assembly/structure 40 comprising a plurality of leaflets 42 situated at least partially within the frame 20. The skirt/covering 112 can prevent the leaflets 91 or portions thereof from extending through spaces between the struts of the frame 20 when the prosthetic valve 110 is crimped, thereby reducing damage to the prosthetic leaflets due to pinching of the leaflets between struts.

The outer skirt/covering 112 can be configured to cushion/protect native tissue (e.g., valve tissue 91) in contact with the prosthetic valve 110 after implantation, and to reduce damage to the tissue due to movement or friction between the tissue and surface(s) of the valve 110. The skirt/covering 112 can also serve to reduce paravalvular leakage. In some examples, the skirt/covering 112 includes a backing layer 119 and a second cushioning layer 118, wherein the cushioning layer can comprise a soft/plush surface oriented radially outward so as to protect tissue or objects in contact with the cushioning layer. In the illustrated configuration, the skirt/covering 112 also includes an atraumatic inflow protective portion 120 extending circumferentially around the inflow end 102 of the frame 20, and an atraumatic outflow protective portion 122 extending circumferentially around the outflow end 101 of the frame 20. The cushioning portion 118 of the skirt 112 can be disposed between the inflow and outflow protective portions 120, 122.

In implementations of the skirt/covering 112 as comprising a backing layer 119 and an outer cushioning layer 118, the backing layer may comprise a material such as gauze, polyethylene terephthalate (PET) fabric (e.g., Dacron), polyester fabric, polyamide fabric, or any of various non-woven fabrics, such as felt. In some examples, the backing layer 119 can also comprise a film including any of a variety of crystalline or semi crystalline polymeric materials, such as polytetrafluorethylene (PTFE), PET, polypropylene, polyamide, polyetheretherketone (PEEK), etc. In this manner, the backing layer 119 can be relatively thin and yet strong enough to allow the skirt/covering 112 to be sutured to the frame 20, and to allow the prosthetic valve 110 to be crimped without tearing. The outer cushioning layer(s) can comprise a soft, plush surface 118. For example, the cushioning layer(s) can be made from any of a variety of woven or knitted fabrics, wherein the surface 118 is the surface of a plush nap or pile of the fabric. Exemplary fabrics having a pile include velour, velvet, velveteen, corduroy, terrycloth, fleece, etc.

The skirt/covering 112 can extend around the exterior of the frame 20 adjacent or against the exterior surfaces of the struts of the frame 20. The skirt/covering 112 can have a length that is greater than the length of the frame 20 as measured along a longitudinal axis A of the frame 20. Thus, the skirt/covering 112 can be situated to extend beyond the apices 29 of the frame struts.

One or more layers (e.g., backing layer) of the skirt/covering 112 can have sufficient length in the axial dimension such that end portions/flaps thereof can be folded over the cushioning portion/layer(s) in the manner of a cuff to form the outflow/inflow protective portions 120, 122. Such overlapping layer(s)/portion(s) can be secured to the underlying layer(s)/portion(s) by attachment means, such as sutures 136, adhesive, clips, etc. In this manner, the inflow and outflow protective portions 120, 122 can be constructed such that the proximal and distal end portions of the skirt/covering 112 are at least partially enclosed by one or more backing layers, which can provide desirable strength and resistance to bending to the inflow and outflow protective portions 120, 122 so that they extend along the longitudinal axis A of the valve 110 without bending or otherwise protruding into the inner diameter of the valve (e.g., by bending under their own weight, by blood flow, or by blood pressure). In this manner, the inflow and outflow protective portions 120, 122 minimally impact flow through the prosthetic valve and avoid interfering with the prosthetic valve leaflets, reducing flow disturbances, and potentially reducing the risk of thrombus.

As shown in FIG. 6, when the prosthetic valve device 110 is deployed within the docking device 70 in the native valve 6, the inflow end portion 102 of the prosthetic valve 110 can be positioned above the superior surface of the native valve annulus 106 and spaced from surrounding tissue. However, in other implementations, depending on the axial positioning of the prosthetic valve 110, which can be varied, the inflow protective portion 120 can contact the native leaflets 91 and prevent them from directly contacting the inflow end of the frame. Depending on the diameter of the prosthetic valve 110 at the inflow end, the inflow protective portion 120 can serve to prevent the atrium wall from directly contacting the apices 29 at the inflow end of the frame 20.

The portions of the native leaflets 91 captured between the docking device 70 and the prosthetic valve 110 can be cushioned by the cushion surface 118 of the skirt/covering 112. In certain examples, the soft, compliant nature and texture of the skirt/covering 112 can increase friction between the native leaflets 91 and the prosthetic valve 110. This can reduce relative movement of the native leaflets and the prosthetic valve as the left ventricle expands and contracts, reducing the likelihood of damage to the native leaflets and the surrounding tissue. The skirt/covering 112 can also provide increased retention forces between the docking device 70 and the prosthetic valve 110. The plush, compressible nature of the skirt cushioning 118 can also reduce protrusion of the skirt/covering 112 through the openings in the frame 20 caused by application of pressure to the skirt/covering 112, thereby reducing interference with the hemodynamics of the valve. Additionally, the outflow cushioning portion 122 can protect the chordae tendineae 18 from contacting the outflow end of the frame 20, thereby reducing the risk of injury or rupture of the chordae.

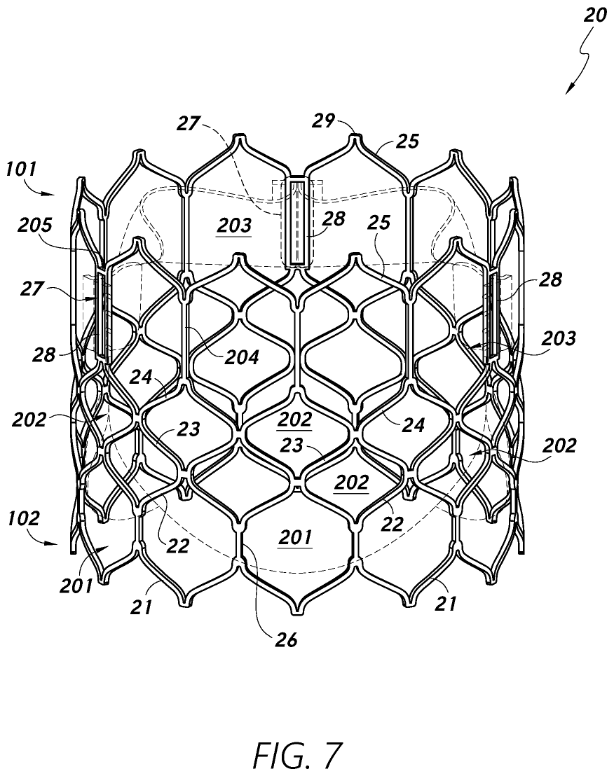

Any of the prosthetic valves described herein may include a frame 20, as referenced and described above. FIG. 7 shows a perspective view from an outflow end of an annular frame 20 of a prosthetic heart valve in accordance with one or more examples.

Referring to FIG. 7, the frame 20 in the illustrated example comprises a first, lower row of angled struts 21 arranged end-to-end and extending circumferentially at the inflow end 102 of the frame; a second row of circumferentially extending, angled struts 22; a third row of circumferentially extending, angled struts 23; a fourth row of circumferentially extending, angled struts 24; and a fifth row of circumferentially extending, angled struts 25 at the outflow end 101 of the frame. A plurality of substantially straight axially extending struts 26 can be used to interconnect the first-row struts 21 with the second-row struts 22. The fifth-row struts 25 can be connected to the fourth-row struts 24 by a plurality of axially-extending window frame portions formed by pairs of axially-extending struts 28 (which define commissure windows 27), as well as by other axially-extending struts 204. Each axial strut 28, 204 and each frame portion 27 extends from a location defined by the convergence of the lower ends of two angled struts 24 to another location defined by the convergence of the upper ends of two angled struts 25.

Each commissure window frame portion 27, which comprises a plurality of vertical struts 28 and/or lateral/horizontal struts 205, is configured for mounting a respective commissure of the leaflet structure 40. Either of the reference numbers 27, 28 can be used herein to refer to the struts forming the commissure cells/windows, or to the commissure cells/windows themselves (e.g., the negative space of the frame 20 defined by one or more of the struts 28, 205). As can be seen, each commissure window 27 is secured at its upper and lower ends to the adjacent rows of struts to provide a robust configuration that enhances fatigue resistance under cyclic loading of the valve compared to alternative cantilevered struts for supporting the commissures of the leaflet structure. This configuration enables a reduction in the frame wall thickness to achieve a smaller crimped diameter of the valve. In particular examples, the thickness of the frame 20 measured between the inner diameter and outer diameter can be less than about 0.5 mm.

The struts and frame portions of the frame 20 collectively define a plurality of open cells of the frame. For example, at the inflow end 102 of the frame 20, struts 21 and struts 22 can define a lower row of cells defining openings 201. The second 22, third 23, and fourth 24 rows of struts can define two intermediate rows of cells defining openings 202. The fourth 24 and fifth 25 rows of struts, along with frame struts 28, 204, can define an upper row of cells defining openings 203. The openings 203 can be relatively large and sized to allow portions of the leaflet structure 40 to protrude, or bulge, into and/or through the openings 203 when the frame 20 is crimped in order to minimize the crimping profile.

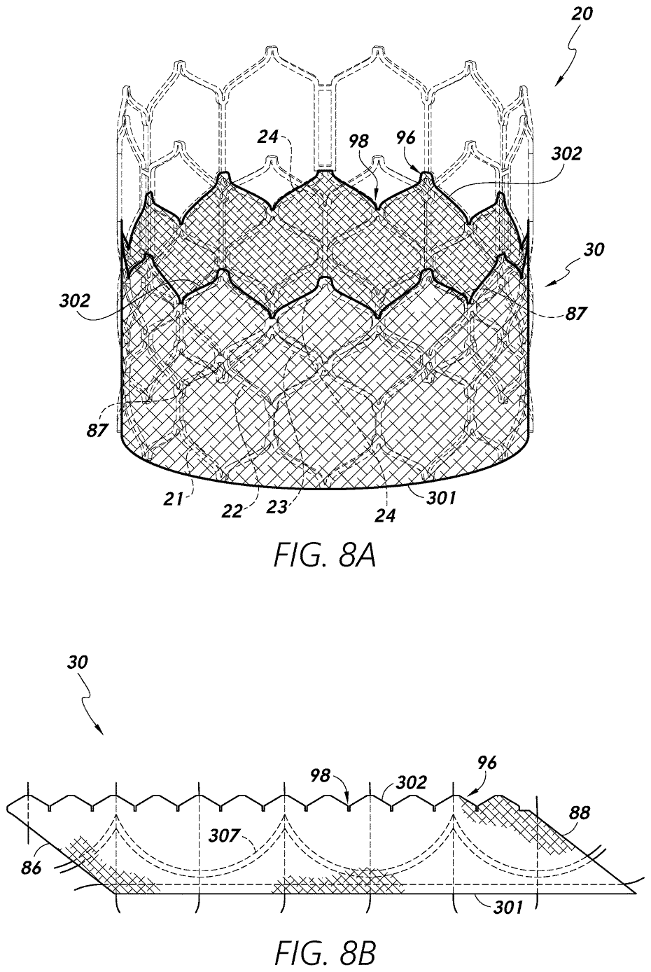

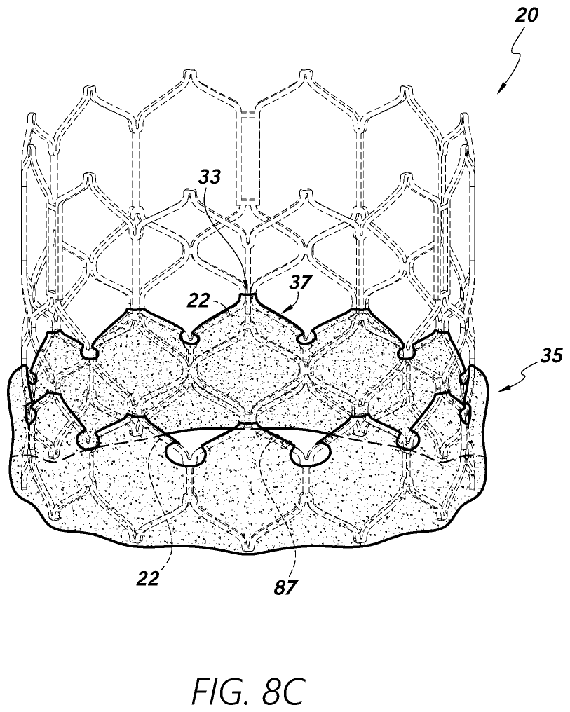

FIG. 8A shows/highlights a main skirt 30 that is disposed, or configured to be disposed, on a frame 20 of a prosthetic heart valve in accordance with one or more examples. FIG. 8B shows the main skirt 30 of FIG. 8A in a flattened/plan view in accordance with one or more examples. FIG. 8C shows/highlights an outer skirt 35 that is disposed, or configured to be disposed, on the valve frame 20 in accordance with one or more examples.

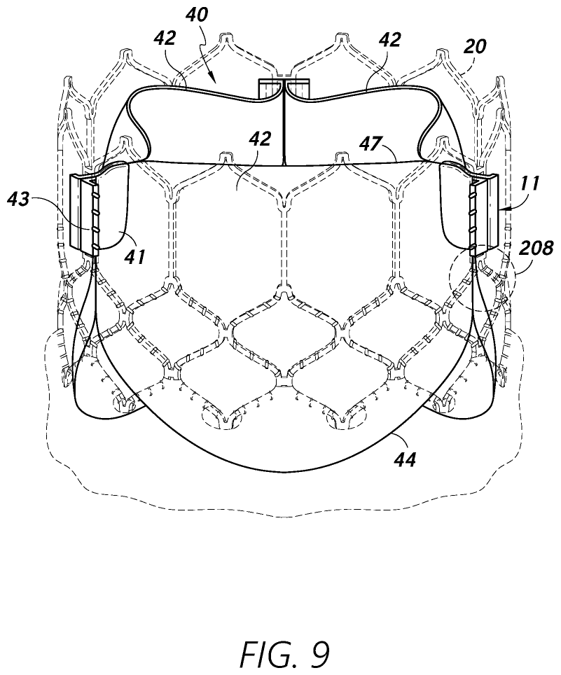

The main functions of the main skirt 30 can be to assist in securing the a leaflet assembly/structure 40 (see FIG. 9, described in detail below) to the frame 20 and to assist in forming a desirable seal between the valve and the native annulus by blocking the flow of blood through the open cells of the frame 20 below the lower edge of the leaflets. The main skirt 30 can desirably comprise a tough, tear-resistant material such as polyethylene terephthalate (PET), although various other synthetic or natural materials can be used. The thickness of the skirt 30 can advantageously be less than 6 mil, and desirably less than 4 mil, such as less than about 2 mil. In some examples, the skirt 30 can have a variable thickness. For example, the skirt 30 can be thicker at its edges than at its center. The thinner portions towards the center of the main skirt 30 can provide for better crimping performances while still providing good perivalvular sealing.

The skirts, or skirt portions, 30, 35 can be secured to the frame 20 via sutures 87 (see FIG. 1). In some examples, the skirt(s) 30/35 comprises woven fibers. FIG. 8B shows a flattened view of the skirt 30 prior to its attachment to the frame 20. The skirt(s) 30/35 can be laser cut or otherwise formed from a strong, durable piece of material, such as woven PET, although other synthetic or natural materials can be used. The skirt 30 can have a substantially straight lower edge 301 and an upper/outflow edge 302 defining a plurality of alternating projections 96 and notches 98. The opposing short edges 86, 88 of the skirt 30 can be configured to be joined together to form the cylindrical/annular shape shown in FIG. 8A. The edges 86, 88 can be non-perpendicular to the upper and lower edges 302, 301, or can be perpendicular in some implementations. In the illustrated configuration, the skirt 30 has a shape resembling that of a rhomboid.

The upper edge portion 302 of the skirt 30 can be formed with a plurality of projections 96 that define an undulated shape that generally follows the shape of the fourth row of struts 24 of the frame 20 (see FIG. 7) immediately adjacent the lower ends of axial struts 204. In this manner, the upper edge of skirt 30 can be tightly secured to struts 24 with sutures 87. The skirt 30 can also be formed with slits/notches 98 to facilitate attachment of the skirt 30 to the frame 20. The slits 98 can be dimensioned so as to allow an upper edge portion 302 of skirt to be partially wrapped around the frame struts 24 and reduce stresses in the skirt 30 during the attachment procedure. For example, the skirt 30 can be placed on the inside of the frame 20 and an upper edge portion of the skirt can be wrapped around the upper surfaces of struts 24 and secured in place with sutures 87. Wrapping the upper edge portion of the skirt 30 around struts 24 in this manner can provide a relatively strong and durable attachment of the skirt 30 to the frame 20. The skirt 30 can further be secured to the first 21, second 22, and/or third 23 rows of struts with sutures 87.

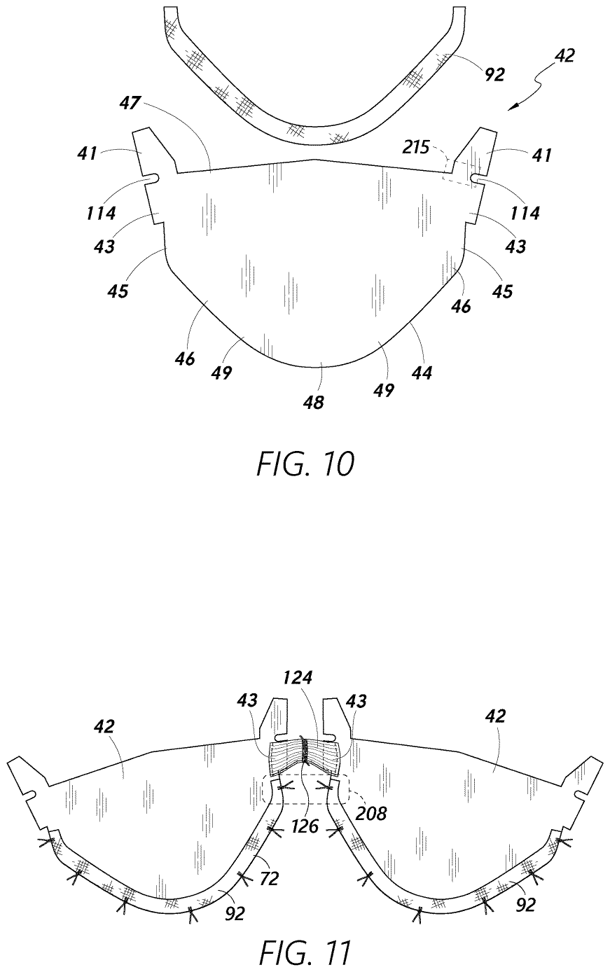

As shown in FIG. 8B, the skirt can be configured to be coupled to valve leaflet edges along an undulating, curved/scalloped suture line 307, which advantageously tracks the scalloped shape of the leaflet structure 40 (see FIG. 9). The skirt 30 can be secured to the leaflet structure 40 through suture-coupling with a protective strip 92 (see FIG. 10) associated with a lower edge/base of the leaflets 42 of a leaflet structure 42. Sutures used to couple the skirt 30 to the frame 20 and/or leaflet structure 40, as with any other sutures described herein, can be any suitable suture, such as an Ethibond suture. The main skirt 30 can be sutured to the frame 20 at locations away from the suture line 307 so that the skirt 30 can be more pliable in such area. This can avoid stress concentrations at the suture line 307, which attaches the lower edges of the leaflets to the skirt 30. The sutures used for coupling the skirt 30 to the leaflet structure 40 can be in-and-out sutures extending through each leaflet 42, the skirt 30, and each reinforcing strip 92.

The lower edge 301 of the skirt 30 can be sutured to the lower edge of the outer skirt/portion 35 at the inflow end 102 of the valve. With respect to the outer skirt 35, each projection 37 can be sutured to the second row/rung of struts 22 of the frame 20. The peaks/corners 33 of the projections 37 can be folded over respective struts 22 and secured with sutures 87.

The outer skirt 35 can be secured to the frame 20 such that when the frame 20 is in its expanded state, there is excess material or slack between the outer skirt's lower and upper edges that does not lie flat against the outer surface of the frame 20. In other words, the outer skirt 35 can be configured with excess material which causes the outer skirt to bulge outwardly as the frame foreshortens (i.e., shortens in length) during radial expansion. Accordingly, when the valve is deployed within the body, the excess material of the outer skirt 35 can fill in gaps between the frame 20 and the surrounding native annulus to assist in forming a fluid-tight seal between the valve and the native annulus. The outer skirt 35 therefore can cooperate with the main skirt 30 to avoid perivalvular leakage after implantation of the valve 10. In addition, the slack between the lower and upper edges of the outer skirt 35 can allow the frame 20 to elongate axially during crimping without any resistance from the outer skirt, wherein the outer skirt 35 does not substantially add to the outer diameter of the prosthetic valve in the crimped condition.

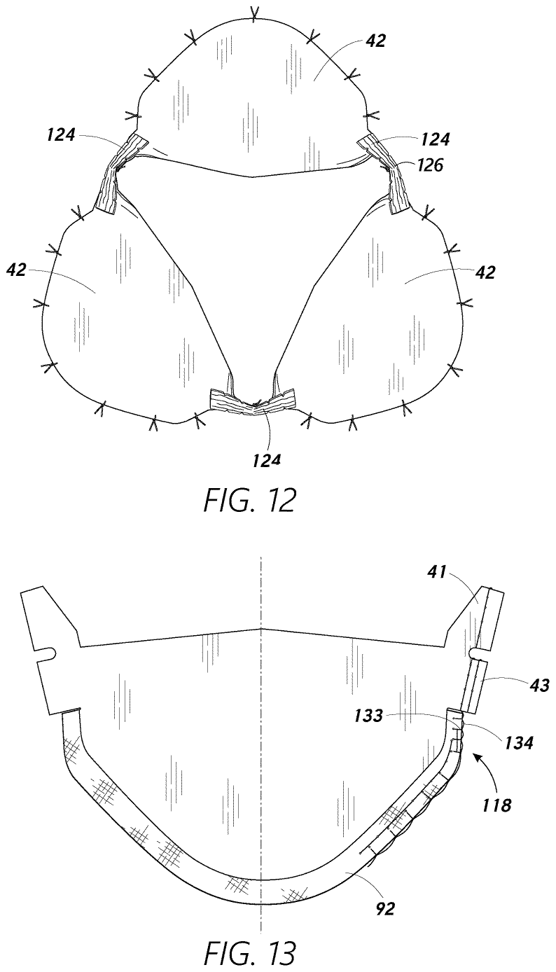

FIG. 9 shows a leaflet assembly/structure 40 of a prosthetic heart valve in accordance with one or more examples. FIG. 10 is a simplified plan view of a single leaflet 42 of the leaflet assembly/structure 40. FIG. 11 is a simplified plan view of a pair of coupled leaflets 42 of a leaflet assembly/structure 40 of a prosthetic heart valve in accordance with one or more examples. FIG. 12 shows leaflets 42 of the leaflet assembly/structure 40 coupled at tab portions 41 in accordance with one or more examples. FIG. 13 shows a leaflet 42 of a valvular assembly/structure having a reinforcing strip 92 and sub-commissure suturing/leaflet-union 118 in accordance with one or more examples.

The description below relating to valve leaflets and leaflet attachment features can be understood with reference to any of FIG. 9-13. The leaflet structure 40 in the illustrated example includes three flexible leaflets 42, although it should be understood that more or fewer leaflets can be used. Each leaflet 42 in the illustrated configuration has an upper (outflow) free edge 47 extending between opposing upper tabs 41 on opposite sides of the leaflet. In some implementations, a notch 114 can be formed below each upper tab 41 that separates the upper tab 41 from a corresponding lower tab 43. The portion 215 of the leaflet 42 adjacent to the notch 114 becomes a folded edge when the upper tab 41 is folded down against/toward the lower tab 43 (see FIG. 15).

In some implementations, the lower (inflow) edge portion 44 of the leaflet extending between respective ends of the lower tabs 43 includes vertical, or axial, lateral edge portions 45 on opposites of the leaflets 42 extending downwardly from corresponding lower tabs 43 and a substantially V-or U-shaped, intermediate edge portion 46 having a smooth, curved apex portion 48 at the lower end of the leaflet and a pair of oblique portions 49 that extend between the axial edge portions and the apex portion. The oblique portions 49 can have a greater radius of curvature than the apex portion 48. Each leaflet 42 can have a reinforcing strip 92 secured (e.g., sewn) to the inner surface of the lower edge portion 44, as shown in FIG. 11. The portion 208 of the leaflets 42 immediately below the laterally-projecting tabs 43 and above the curved lower edge/cusp 44 may be considered the sub-commissure portions 118 of the leaflets 42. For example, the contour of the leaflets 42 below the commissure tabs 43, 41 may follow the more-vertical/straight lateral sides/edges 45, leading to the more-curved/circular lower cusp edge 44. With regard to valve leaflets, references herein to leaflet ‘edges’ can be understood to refer to terminal edges/surfaces of a leaflet portion (e.g., terminal surface that is perpendicular to a plane of the leaflet in the area of the portion), and/or to the leaflet material/surface immediately adjacent the terminal edge (e.g., less than 2 mm, or less than 1 mm) that lies in-plane with the leaflet.

The leaflet/valvular structure 40 can be attached to the skirt 30 (see FIG. 8A) via one or more thin reinforcing strips 92 (e.g., PET or other material forming a sleeve), which can advantageously enable a secure suturing and/or protect the, e.g., pericardial tissue of the leaflets 42 from tears. The leaflet/valvular structure 40 can be sandwiched between the main skirt 30 and the reinforcing strip(s) 92. Sutures can be implemented that track the curvature of the bottom edge 44 of the leaflet structure 40 and the suture line 307 along the skirt 30 (see FIG. 8B).

The leaflets 42 can be secured to one another at their adjacent sides to form commissures 11 of the leaflet structure 40. A plurality of flexible connectors 124, which may have the form of a sheet, strip, or tab, can be used to interconnect pairs of adjacent sides of the leaflets 42 and to mount the leaflets to commissure window frame portions 27 (see FIG. 7). The flexible connectors 124 can be made from a piece of woven PET fabric, although other synthetic and/or natural materials can be used. Each flexible connector 124 can include a wedge 126 extending from the lower edge to the upper edge at the center of the connector. The wedge 126 can comprise a non-metallic material, such as a rope or a piece of Ethibond 2-0 suture material, secured to the connector with, for example, a temporary suture. The wedge 126 can help prevent rotational movement of the leaflet tabs once they are secured to the commissure window frame portions 27. The flexible connector(s) 124 may be stitched or otherwise coupled to the outer sides/surfaces of the tabs 43, with a gap/space between the edges of the tabs 43 spanned by a medial portion (with respect to the lateral/longitudinal dimension of the illustrated orientation of FIG. 11) of the connector(s) 124. As used herein, the outer surface/side of the leaflets 42 and/or component(s) thereof is considered the side/surface of the leaflets 42 facing the reader in FIGS. 10 and 11.

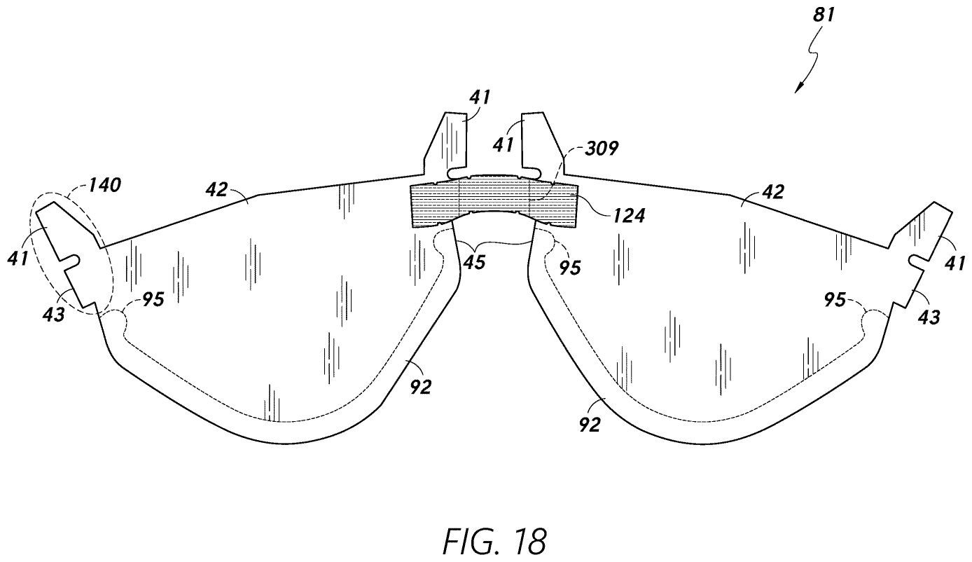

FIG. 11 shows the adjacent sides of two leaflets 42 interconnected by a flexible connector 124. The opposite end portions of the flexible connector 124 can be placed in an overlapping relationship with the lower tabs 43. Each tab 43 can be secured to a corresponding end portion of the flexible connector 124 by suturing. Three leaflets 42 can be secured to each other in a side-to-side disposition using three flexible connectors 124, as shown in FIG. 12. While FIG. 11 shows the reinforcing strips 92 on the same sides of the leaflets 42 (e.g., both on the outer surface/sides of the leaflets) as the connectors 124, in some implementations, the flexible connector 124 may be implemented on an outer surface/side of the leaflets 42, whereas the lower cusp strips 92 may be implemented additionally or alternatively on the inner surface of the leaflets 42 (as in FIGS. 18-23).

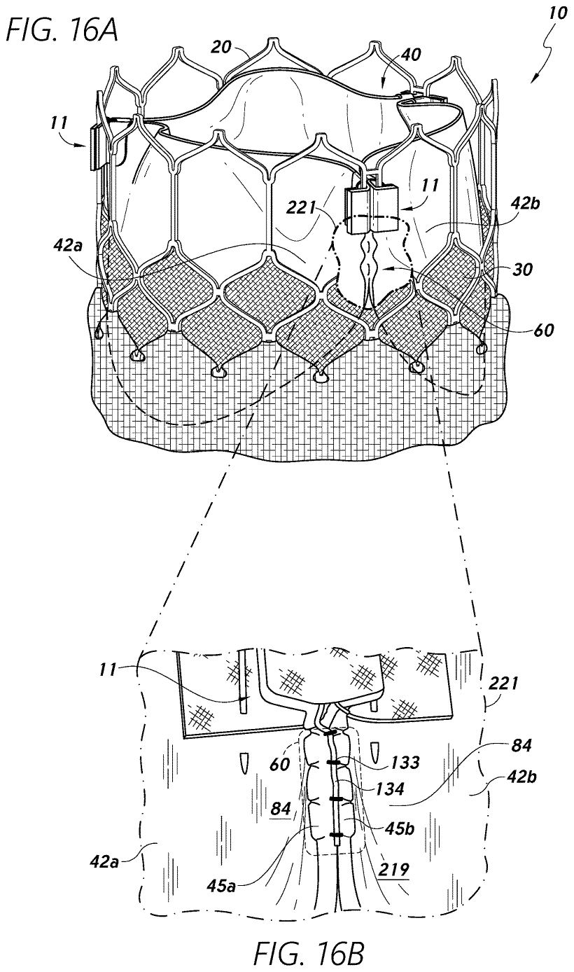

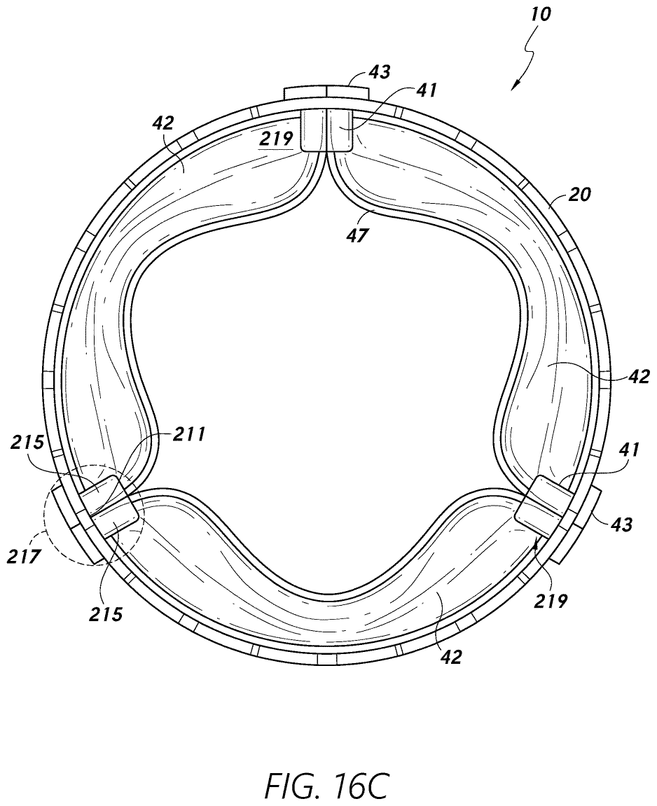

Referring to FIG. 13 (also relevant to FIGS. 16A and 16B, described in further detail below), adjacent sub-commissure portions 118 of two leaflets 42 can be sutured directly to each other. In the example shown, suture material (e.g., PTFE-6-0) is used to form in-and-out stitches 133 and comb stitches 134 that extend through the sub-commissure portions 118 and the reinforcing strips 92 on both leaflets 42. The two remaining pairs of adjacent sub-commissure portions 118 can be sutured together in the same manner to form the assembled leaflet structure 40, which can then be secured to the valve frame 20.

As noted above, the main skirt 30 can be used to assist in suturing the leaflet structure 40 to the frame 20. The skirt 30 can have an undulating temporary marking suture in the area of the suture line 307 to guide the attachment of the lower edges 44 of each leaflet 42. The skirt 30 itself can be sutured to the struts of the frame 20 using sutures 87, as noted above, before (or after) securing the leaflet structure 40 to the skirt 30. The struts that intersect the suture line 307 may not ultimately be attached to the skirt 30. This can allow the skirt 30 to be more pliable in the areas not secured to the frame 20 and can minimize stress concentrations along the suture line 307 that secures the lower edges 44 of the leaflets 42 to the skirt 30.

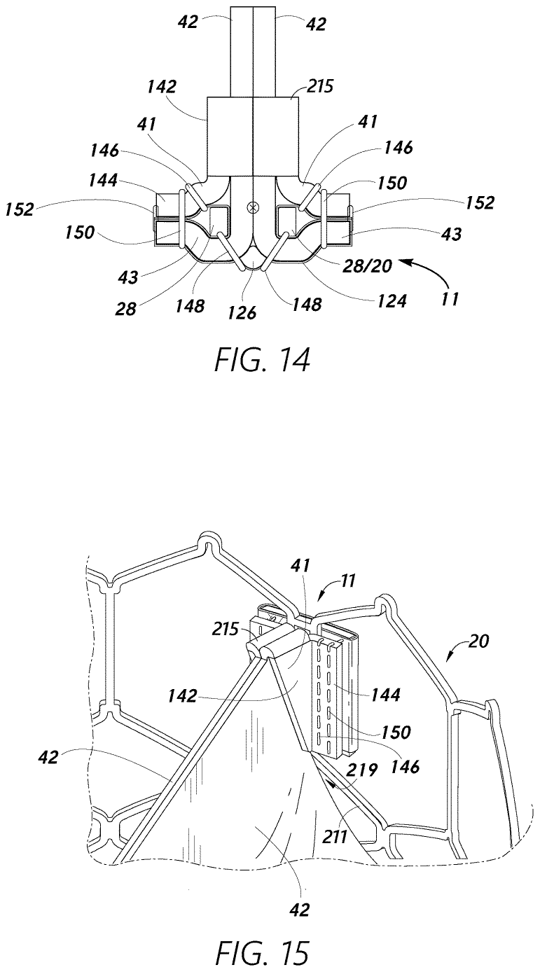

FIG. 14 shows a simplified top-down (possibly cross-sectional) view of a commissure portion 11 of a valvular/leaflet structure 40 attached to an annular frame 20 of a prosthetic heart valve in accordance with one or more examples. FIG. 15 is a close-up perspective view of the commissure portion 11 attached to the frame 20 from a perspective/vantagepoint within the frame 20 looking radially outwardly. In the provided images, adjacent tab portions 41, 43 of two leaflets 42 are shown secured to a corresponding window frame portion 27 defined by frame struts 28.

With reference to any of the previously-described figures, the flexible connector 124 securing two adjacent sides of two leaflets 42 can be folded widthwise, with the upper tab portions 41 folded downwardly against the flexible connector 124. As best demonstrated in FIG. 15, when coupling the leaflets 42 to the commissure window 27 of the frame, each upper tab portion 41 of the leaflets 42 can be creased lengthwise (vertically) to assume an L-shape having an inner portion 142 folded against the inner surface of the leaflet 42 and an outer portion 144 folded against the connector 124. The outer portion 144 can then be sutured to the connector 124 along a suture line 146. The commissure tab assembly (comprised of a pair of lower tab portions 43 connected by connector 124) can be inserted through the commissure window 27 of a corresponding window frame portion 28. FIGS. 14 and 15 show the commissure tab assembly 11 extending outwardly through the window frame portion 27 of the frame 20.

With reference to FIG. 14, the commissure tab assembly 11 can be pressed radially inwardly at the wedge 126 such that one of the lower tab portions 43 and a portion of the connector 124 is folded against the frame 20 on one side of the window frame portion 28 and the other lower tab portion 43 and a portion of the connector 124 can be folded against the frame 20 on other side of the window frame portion 28. A pair of suture lines 148 can be formed to retain the lower tab portions 43 against the frame 20 in the manner shown in FIG. 14. Each suture line 148 can extend through the connector 124, a lower tab portion 43, the wedge 126, and another portion of connector 124. Then, as shown in 14, each lower tab portion 43 can be secured to a corresponding upper tab portion 41 with a primary suture line 150 that extends through one or more layers of the connector 124, the lower tab portion 43, and the upper tab portion 41. Finally, as shown in FIG. 14, the suture material used to form the primary suture line 150 can be used to further form whip stitches 152 at the edges of the tab portions 41, 43 that extend through two layers of connector 124 sandwiched between the tab portions 41, 43.

After all three commissure tab assemblies 11 are secured to respective window frame portions 27, the lower edges 44 of the leaflets 42 between the commissure tab assemblies 11 can be sutured to the main skirt 30 along the suture line 307. The sutures can be in-and-out sutures extending through each leaflet 42, the skirt 30 and each reinforcing strip 92. The leaflets 42 can be further secured to the skirt 30 with blanket sutures that extend through each reinforcing strip 92, leaflet 42, and the skirt 30 while looping around the edges of the reinforcing strips 92 and leaflets 42.

### Leaflet Opening Dynamics

As shown in FIGS. 4 and 6 and described above, in some implementations, a prosthetic heart valve, such as the heart valve 10 described above, can be implanted within a native heart valve to help prevent or inhibit retrograde flow/regurgitation and/or to address other insufficiency of the native heart valve. In other implementations, a prosthetic heart valve as described herein can be implanted in a blood vessel that leads to a heart chamber, such as the inferior vena cava 16 or the superior vena cava 19, to prevent or inhibit backflow therein during the systolic phase.

In general, prosthetic heart valves implanted at the aortic position (e.g., within the native aortic valve 7, as shown in FIG. 4) can normally experience relatively-high pressure gradients (e.g., driving pressure of about 125 mmHg). In contrast, prosthetic heart valves implanted at the pulmonary position (e.g., within the native pulmonary valve), the tricuspid position (e.g., within the native tricuspid valve 8), the mitral position (e.g., within the native mitral valve 6), or blood vessels leading to heart chambers (e.g., within the inferior vena cava 16 or superior vena cava 19) can normally experience relatively-low pressure gradients (e.g., driving pressure of about 30 mmHg or less).

With particular relevance to examples disclosed herein, a prosthetic heart valve may be exposed to a relatively-low pressure hemodynamic environment (e.g., where the driving pressure that causes the implanted valve to open is less than 30 mmHg) when implanted in the atrioventricular valves, such as within the mitral valve 6 as shown in FIG. 6. In conventional prosthetic heart valves, the low pressure gradient may cause abnormal motion of the leaflets as the valve transitions between open and closed configurations of the leaflet assembly/structure. The leaflet motion abnormalities can result in stasis, which in-turn can make the leaflets more susceptible to chronic thrombosis, thickening (which further restricts the motion of the leaflets), or both.

In some implementations, coupling of the leaflets of the leaflet assembly/structure of a prosthetic valve to the frame and/or to one another can affect the compliance/responsiveness of the valve leaflets, that is, the natural tendency of the leaflets to open under relatively low pressure conditions rather than requiring a substantial pressure gradient to produce leaflet opening. Disclosed herein are leaflet coupling examples, such as with respect to sub-commissure leaflet-to-leaflet coupling, that are designed to avoid, or at least reduce, the incidence of stasis when implanted at a relatively-low pressure gradient hemodynamic location by promoting/increasing the compliance/responsiveness of the valve leaflet assembly.

### Leaflet-To-Leaflet Edge Coupling

The present disclosure relates to example leaflet structures that have sub-commissure leaflet-to-leaflet coupling configurations that promote leaflet openness, such that the leaflets are more naturally prone to opening and have a tendency to open under relatively low pressure gradients as may be present in, for example, mitral valve positions. Such valve openness/compliance can be achieved at least in part through implementation of leaflet edge-to-edge coupling wherein the adjacent leaflet edges are directed radially inward with respect to the axis of the leaflet structure and/or valve. Examples of the present disclosure provide alternative leaflet-to-leaflet edge coupling solutions compared to other implementations in which leaflet edges are coupled with the leaflet edges oriented radially outward where joined together as shown in the example of FIGS. 16A-C.

FIG. 16A shows a prosthetic heart valve 10 having a valvular/leaflet structure 40 with leaflets 42 including outwardly-oriented edge-to-edge leaflet unions 60 in accordance with one or more examples. FIG. 16B shows sub-commissure portions of adjacent leaflets 42a, 42b of the leaflet/valvular structure 40 of the prosthetic heart valve 10 of FIG. 16A with leaflet edge portions 45a, 45b joined in a radially-outward orientation. FIG. 16C shows an axial view from an outflow end of the prosthetic heart valve 10 of FIGS. 16A and 16B in an open configuration in accordance with one or more examples.

Referring now to FIG. 16A-C, the adjacent sub-commissure edges/portions 45a, 45b of two leaflets 42a, 42b can be sutured directly to each other. In the example shown, suture material (e.g., PTFE-6-0) is used to form in-and-out stitches 133 and comb stitches 134 that extend through the sub-commissure portions/edges 45. The sutures/stitches 133, 134 may further pass through reinforcing strips 92 (not visible in FIGS. 16A-C) on the backside of both leaflets 42a, 42b. The two remaining pairs of adjacent sub-commissure portions/edges 45 associated with the other two commissures 11 of the valve 10 can be sutured together in the same manner to form the assembled leaflet structure 40, shown secured to the frame 20 in FIG. 16A. With the leaflet edges 45a, 45b oriented outward, the leaflet tissue 84 adjacent to the edges 45a, 45b can be forced inward, away from the inside surface 211 of the frame 20, thereby forming a gap 219 between the leaflets 42 and the inside surface 211 of the frame in the sub-commissure areas 221.

As illustrated in FIG. 16C, the outward orientation of the leaflet edges 45a, 45b (not visible in FIG. 16C; see FIGS. 16A, 16B) can inhibit opening of the leaflets 42, such that in the open configuration as shown in FIG. 16C, the leaflets 42 do not readily open in low-pressure-gradient conditions, or open wide enough to expose the inner surface 211 of the frame 20 in the area of the leaflet-to-leaflet union 217. For example, adjacent tab folds 215 in the leaflet union area 217 may generally remain in a generally parallel/abutting position as in FIG. 16C rather than separating/splaying apart to expose the frame 211 when the leaflet structure 40 opens.

### Inverted Leaflet-To-Leaflet Edge Coupling to Promote Leaflet Opening

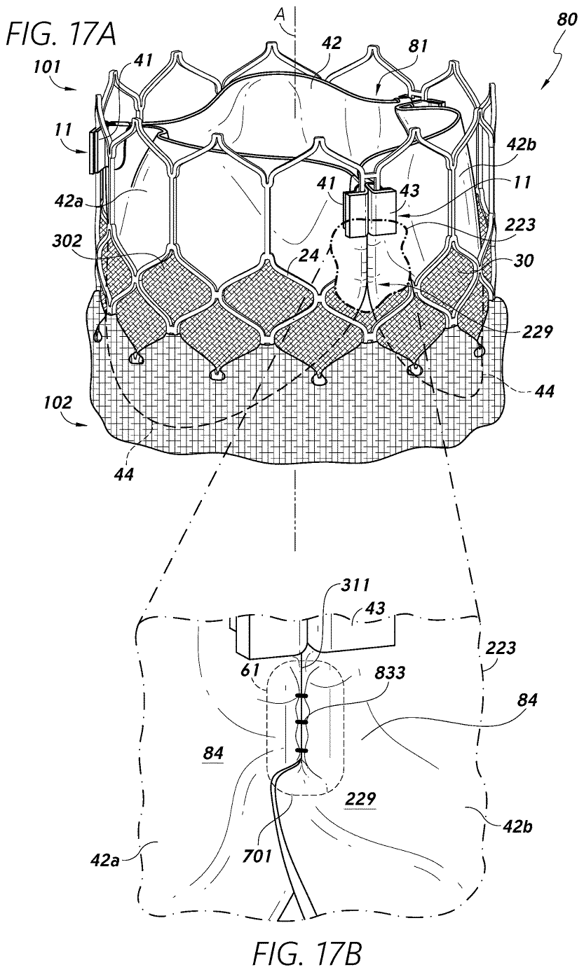

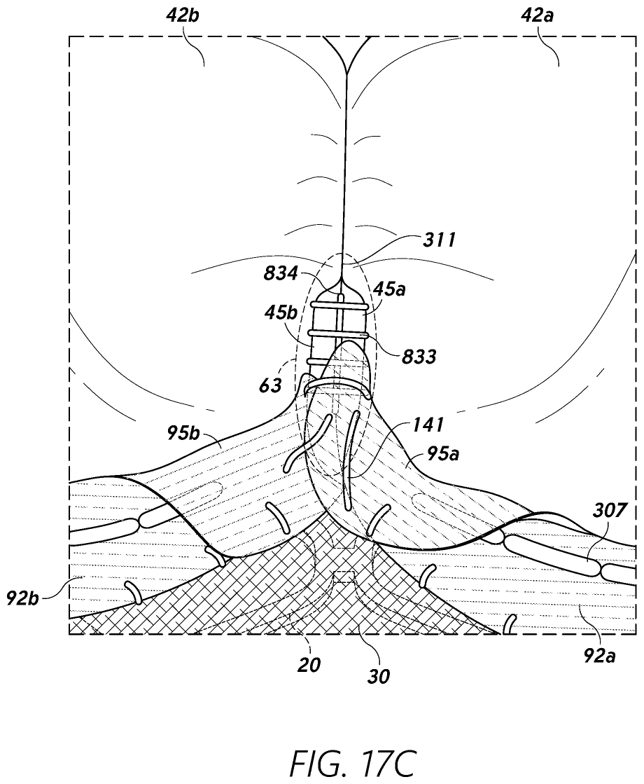

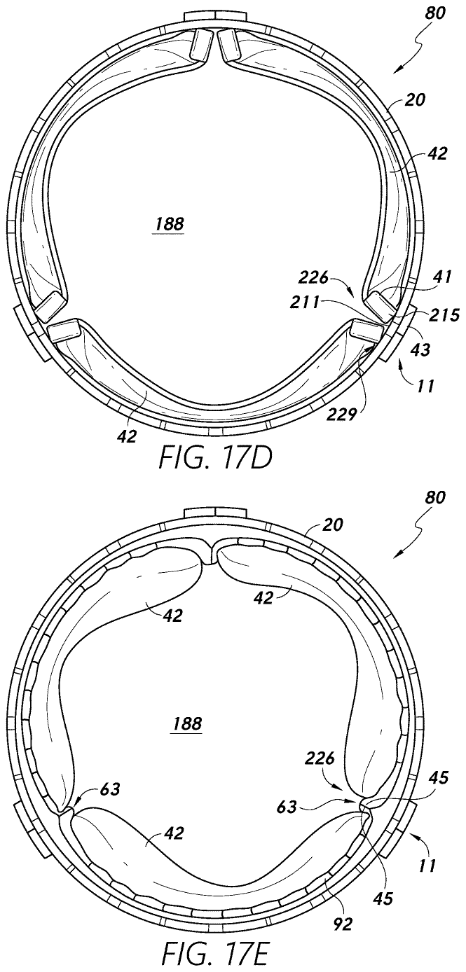

In contrast to the example of FIG. 16A-C, certain examples disclosed herein provide leaflet/valvular structures that are configured to transition between open and closed configurations relatively more easily, thereby avoiding abnormal leaflet motion in low-pressure gradient implant locations. For example, the implementation of inwardly-oriented leaflet edge coupling, as described in detail below, can promote full opening of valve leaflets under relatively low-flow and/or pressure gradient conditions. By implementing inwardly-oriented leaflet edge coupling as disclosed herein, the gap between the inside surface of the valve frame and the leaflets in the sub-commissure area can be reduced, which can promote the predisposition of the leaflets in a naturally more open state and assist with washout of the neo-sinus region. Such neo-sinus washout together with the avoidance of leaflet stasis in low-pressure implantation environments can further reduce the risk of thrombosis. In some implementations, the easier transition to the open leaflet configuration may also result in a larger outlet open area 188 of the valve, as shown in FIG. 17D.

FIG. 17A shows a prosthetic heart valve 80 having a leaflet/valvular structure 81 with leaflets 42 including inwardly-oriented edge-to-edge leaflet unions 61, which may be referred to as ‘leaflet-to-leaflet’ unions, in accordance with one or more examples. FIG. 17B shows an outer view of a sub-commissure portion 223 including joined adjacent leaflets 42a, 42b of the leaflet/valvular structure 81 of the prosthetic heart valve 80 of FIG. 17A with leaflet edges 45a, 45b joined in a radially-inward orientation. FIG. 17C shows an inner view of the sub-commissure portion of the leaflet/valvular structure 81 shown in FIG. 17B. FIG. 17D shows an axial view from an outflow end 101 of the prosthetic heart valve 80 of FIG. 17A-17C in an open configuration. FIG. 17E shows an axial view from an inflow end 102 of the prosthetic heart valve of FIG. 17A-17D in an open configuration. As a matter of convenience, in view of the similarity of certain features shown in FIG. 17A-17E to features/components of other examples presented herein, common reference numbers are used in FIG. 17A-E as in other figures of the present disclosure with respect to such similar features. It should be understood that features identified with common reference numbers can have any of the same features/components as other similarly-numbered features, and/or may differ in any respect from other similarly-numbered/referenced features. Therefore, reference identifiers and associated features/components presented herein should be understood primarily in the context of the written description of the particular figure(s) being described, though description of other figures using similar reference numbers/identifiers can provide additional optional details and/or alternatives for such referenced features. Examples of a prosthetic heart valve, disclosed herein or otherwise, can include any of the innovations and variations discussed herein with respect to FIGS. 17A-17E.

In the example of FIG. 17A-17E, sub-commissure edges 45a, 45b of respective adjacent leaflets 42a, 42b can be joined in a manner such that the edges 45a, 45b point inwardly with respect to an axis A of the valve 80 and/or leaflet structure 81, wherein the edges 45a, 45b are joined using a suitable suturing technique or other coupling means or mechanism. For example, the two adjacent leaflets 42a, 42b can be prepared and aligned so that the edges 45a, 45b are deflected radially inwardly to abut one another in an approximately parallel orientation, as shown in FIG. 17C. The alignment of the edges 45a, 45b as shown can help ensure optimal functionality and cohesiveness of the attached leaflets 42a, 42b post-procedure.

With the targeted edges 45a, 45b accurately aligned in a radially-inward direction relative to the heart valve's axis A, a blanket/comb stitch 833 can be employed to secure the leaflets 42a, 42b together. The suture material may be selected to provide desirable strength, flexibility, and/or biocompatibility. The stitch(es) 833 can be applied along the aligned, juxtaposed edges 45a, 45b, resulting in a robust, resilient sutured seam or junction 61. The leaflet-to-leaflet union/seam 61 acts as an integrating medium, allowing the interconnected leaflets 42 to operate as a singular cohesive unit while maintaining their individual structural integrity. The implementation of the seam 61 with inwardly-oriented edges 45a, 45b can provide improved leaflet integration due to the leaflet tissue 84 adjacent to the seam 61 naturally conforming to the generally outwardly convex/cylindrical form of the frame 20 and/or base of the leaflets 42 in the area of, and adjacent to, the union 61, rather than being deflected radially inwardly and presenting a concave outer surface in the area of the leaflet union, as in the example of FIG. 16B. That is, the shape of the leaflet structure 81 may have a more cohesive, continuous form compared to certain other examples, resulting in improved mechanical continuity, reduced stress concentrations, improved load distribution, improved hydrodynamic efficiency, improved fatigue resistance, and/or other mechanical benefits. The leaflet union 61 can promote seamless cooperation of the leaflets 42, and thus contribute to the optimal functioning of the heart valve 80 within its physiological environment. As with other examples disclosed herein, the device 80 of FIGS. 17A-17E can provide enhanced stability and functionality in the attachment of tissue leaflets in the commissure portions of heart valve stent frames.

When leaflet edges are oriented to point radially outwardly in leaflet-to-leaflet unions, as in FIG. 16A-16C, the sections 84 of the leaflets adjacent to such edges can naturally assume a curvature, manifesting as a concavity when observed from the outside. This geometric alignment can inherently bias the leaflet structure towards a closed position due to the resultant internal stresses and mechanical alignment generated within the leaflet tissue. Such closure bias can be attributed to the inclination of the adjacent leaflet sections towards the valve axis, resulting from the outward pointing arrangement of the edges, fostering a tendency for the leaflets to converge or approximate. In such configurations, the valve leaflets can undesirably resist opening in low-pressure environments, and can be limited in the degree to which the leaflets open, as demonstrated in FIG. 16C. The induced concavity and the consequential mechanical predisposition in the configuration where the leaflet edges point radially outwardly can be desirable in some relatively high-pressure environments as promoting reliable and efficient valve closure, thus enhancing the valve's functional integrity and performance within the cardiovascular system. However, in low-pressure environments, such as in mitral valve replacement, predisposition to valve closure can be undesirable.

With reference to the example of FIG. 17A-17E, when leaflet edges 45 are oriented/aligned to point radially inwardly in leaflet-to-leaflet unions, the inherent curvature of the adjacent leaflet sections 84 can be angled away from the valve axis. The potentially divergent mechanical alignment and stress distributions associated with such leaflet couplings can lead to a predisposition for the leaflets to naturally occupy a more-open state, which more readily expands in response to pressure/flow increases. Such configurations can be beneficial for mitral valve replacement applications and other low-pressure environments.

Where a leaflet apparatus of a prosthetic valve is too resistant to leaflet opening, leaflet motion abnormalities can result that can cause stasis, which can lead to chronic leaflet thrombosis and/or thickening. For example, due to contrasting physiological pressure and flow conditions experienced by the aortic and mitral valves, a naturally-open resting position for a prosthetic valve as disclosed herein may provide more effective opening and closing of the valve prosthesis. The inwardly-oriented leaflet joinder of FIGS. 17A-17E can provide for lower opening, or “cracking” pressures. By ensuring regular opening and closing of leaflets implanted in low-pressure environments, such as the mitral position, instances of leaflet thrombosis events can be reduced.

With reference back to FIG. 16A-16B, a comb stitch 134 can be implemented that joins adjacent leaflets 42a, 42b together below (with respect to the illustrated orientation) commissure tabs 11. As referenced above, the comb stitch 134 in FIG. 16A-16B can cause the leaflets 42a, 42b to be predisposed to a more naturally closed state at that location. In the example of FIG. 17A-17B, the stitching of the leaflet edges 45a, 45b is modified in a way that forces the leaflets to be pre-disposed to a naturally open state. Compared to the implementation of FIG. 16A-16C, the joint/union 61 of the valve 80 of FIG. 17A-17E is inverted, such that the hinge point is reversed. In such an implementation, the leaflet structure 81 may be configured to be open without requiring any flow or pressure gradient to fully open and to reduce the gap 229 between the leaflets 42a, 42b and the frame 20 in the open position to assist with washout. The suturing (e.g., comb stitch) 833, 834 can be similar in form in one or more respects to the comb stitching 133, 134 shown and described in connection with FIG. 16B.

In the cut-away view of FIG. 17A, it should be appreciated that the portion 84 of leaflet tissue in and adjacent to the sub-commissure region 223 is positioned close to and/or in contact with the inside of the frame 20 (some of which is cut-away in FIG. 17A to show the relevant leaflet portions). For example, at the level of the leaflet-joining stitch(es) 833, 834 with the inverted design, whereas other examples may include a visible gap 219 in such areas.

The upper/outflow edge 302 of the skirt 30 may conform with the row of struts 24, or may otherwise terminate in the area below the commissures 11. In such configurations, the skirt 30 may cover/overlap the leaflet interconnection/union 61, at least in part, radially outside of the union 61 of the leaflets 42. The leaflet tabs 41, 43 may be coupled to the frame 20 above the upper/outflow end boundary/edge 302 of the skirt and/or the leaflet-to-leaflet union 61. References to components that are positioned ‘above,’ ‘below,’ or have ‘lateral’ or ‘vertical’ dimensions/orientations may be understood with respect to the vertical orientation of the valve 80 shown in FIG. 17A and other figures.

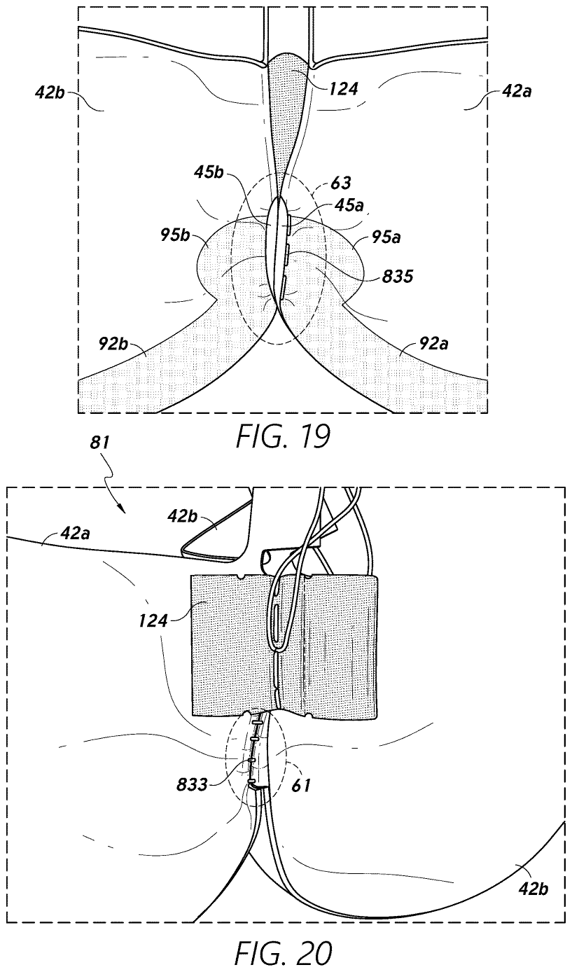

With reference to FIGS. 17B and 17C, which show the exterior and interior views of the inverted comb stitch 61/63 at the leaflet junction/union, respectively, the comb stitch 63 joins the leaflets 42a, 42b together below the coupling of the leaflet tabs 41/43 to the frame 20 at the commissure attachment 11. The comb stitch 61/63 can comprise six, four, or other number, stitches that include a series of lateral, parallel stitches/teeth 833 projecting from and/or coupled to one or more baseline/spine stitches/lines 834. The spine stitch(es) 834 can form a straight stitch that serves as the base from which the “teeth” 833 protrude/project in the radially-outward direction. The stitches 833 may be considered lateral, tooth stitches, such that they may be closer to perpendicular than to parallel with the axis A of the valve 80. The stitching 833/834 can be formed of a straight stitch or a backstitch for more stability. The teeth stitches 833 can be substantially uniform in length, and can be achieved with straight stitches. Any suitable (e.g., consistent) vertical spacing may be implemented between the teeth stitches 833. The union 61/63 of the leaflet edges 45a, 45 b may be implemented as shown such that the edges 45a, 45b are deflected toward an inner side of the leaflets 42a, 42b. The stitch/union 63 (e.g., the interior aspect of the leaflet union/seam 61 shown in FIGS. 17A and 17B) can be formed by placing common outer surfaces/sides of the leaflet edges 45a, 45b and/or adjacent leaflet portions 84 against one another and implementing stitch(es) or other attachment means from the inner/interior sides/surfaces of the leaflet edges 45a, 45b and/or adjacent leaflet portions 84.

Whereas FIGS. 17A and 17B show the exterior/outer portions of the seam/union 61, FIG. 17C shows the internal view of the relevant area, where the inverted comb stitch 63 stitching the leaflet edges 45a, 45b, main skirt 30, and reinforcing strips 92a, 92b come together. References herein to the ‘seam’ of the union 61 can be understood to refer to the line 311 where the leaflets 42a, 42b are held together by the stitching/suture(s) or other coupling means (e.g., adhesive, clip, stable, etc.), or may refer to the stitching/suture(s) or other coupling means. FIGS. 17A and 17B show the outer surfaces of the leaflet tissue 84 adjacent the seam line 311, which leaflet portions/tissue may be substantially flush when held together by the seam/union 61/311. The leaflet portions 84 adjacent the seam 61 and/or edges 45a, 45b can abut each other along the seam line 311. With the leaflet union 61 implemented as an inwardly directed, inverted coupling as shown, the stitches 833, 834 (or stitch(es) 835 in FIG. 19) may be positioned primarily on an interior of the cone/cylinder formed by the leaflets 42 of the leaflet assembly 40, though some of the stitches may be visible and/or at least partially exposed/visible between the leaflet edges 45a, 45b from the exterior view/vantagepoint of FIGS. 17A, 17B, as demonstrated/shown in FIG. 17B. From the area of the comb stitch 63, the leaflets 42a, 42b, together with the reinforcing strips 92a, 92b, are sutured to the main skirt 30 along the scallop line 307. In some implementations the upper portions 701 of the curved cusp edges/portions 44 of the leaflets 42a, 42b may overlap radially and circumferentially, as shown in FIG. 17B just below the leaflet union 61.

The axial views of FIGS. 17D and 17E show the expanded opening area 188 of the modified valve 80. As shown, with the inverted comb stitch 63 implemented with the leaflet edges 45 coupled with an inwardly-pointing orientation, when the leaflets 42 are opened, the tab fold areas 215 may be permitted to separate to a greater degree compared to other examples. Such separation/splaying of the tab folds 215 in the commissure areas can form a gap 226 between the adjacent leaflets 42. In some implementations, the gap 226 may expose the inside 211 of the frame 20. With the leaflet assembly 81 having an open bias, the leaflets 42 may contact the frame, or the main skirt positioned within the inner diameter of the frame. The inner diameter of the valve 80, as shown in FIG. 17E, may include some leaflet edge protrusion, which may have some minor effect on fluid dynamics through the valve.

FIG. 18-25 provide images demonstrating example steps and states/configurations associated with the formation of leaflet assemblies/structures and/or the attachment thereof to frame, skirt, and/or other components of a valve device, in accordance with some example implementations. FIG. 18 shows adjacent leaflets 42 of a valvular/leaflet structure 81 coupled at respective lateral commissure tab portions 41 in accordance with one or more examples of the present disclosure. The lower tabs 43 of the adjacent sides of the two leaflets 42 can be interconnected by a flexible connector 124. For example, the opposite end portions of the flexible connector 124 can be placed in an overlapping relationship with the lower tabs 43. Each tab 43 can be secured to a corresponding end/side portion of the flexible connector 124 by suturing along a line 309. Though only two leaflets 42 are shown in FIG. 18, all three leaflets (for three-leaflet implementations) can be coupled using respective lower tabs and connectors in the manner shown in FIG. 18. The tabs 41, 43 may be considered lateral commissure tabs or tab portions 140.

Once the leaflets 42 have been coupled using the flexible connectors 124 (e.g., PET cloth, or similar), the leaflet assembly/structure 81 may be turned inside-out to accommodate execution of the inverted comb stitch 63 to join the leaflet edges 45. FIG. 19 shows an inner view of leaflets 42a, 42b of the leaflet assembly/structure 81 joined at respective sub-commissure portions/edges 45a, 45b in a radially-inward/inverted configuration in accordance with one or more examples. In FIG. 19, the coupling/suturing 835 is shown as a straight seam transverse through the edges 45a, 45b to provide an additional example implementation for executing an inverted leaflet edge coupling in accordance with aspects of the present disclosure. Additionally or alternatively, the coupling/suturing 835 may be implemented as a comb stitch as shown in FIG. 17C. In some implementations, at least one of the stitches of the inverted stitch 835 (e.g., comb stitch or straight seam stitch) passes through the main skirt of the valve, such that the sub-commissure portion/union 63 of the leaflets is coupled directly to the main skirt. FIG. 20 shows an outer view of the leaflets 42a, 42b of the leaflet assembly 81 joined in the radially-inward/inverted union 61/63 (e.g., comb stitch).

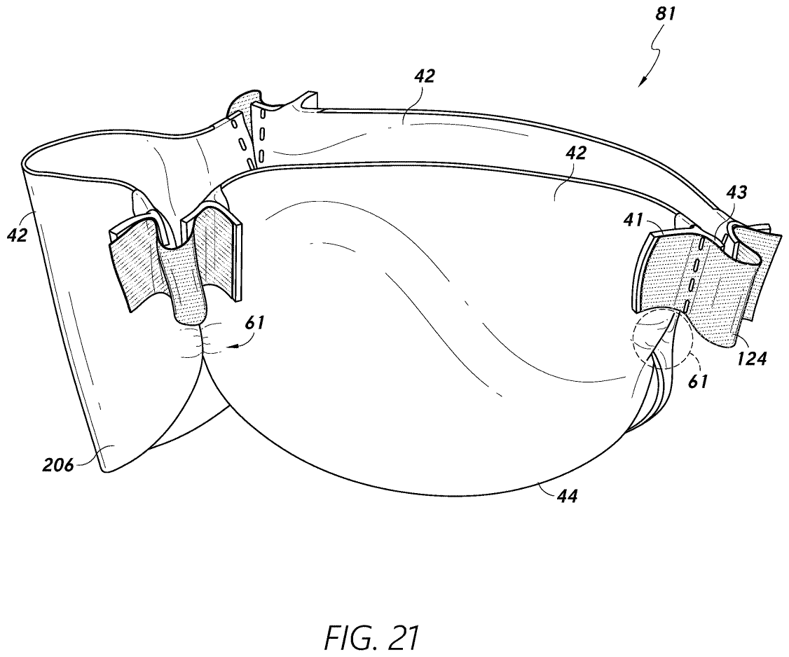

FIG. 21 shows the leaflet assembly/structure 81 turned fully back right-side-out with the attached connectors 124 and inverted leaflet-to-leaflet sub-commissure unions/couplings 61. FIG. 21 shows all three leaflets 42 of the three-leaflet assembly 81, though it should be understood that aspects of the present disclosure may be implemented with leaflet assemblies comprising more or fewer leaflets. When coupled together as in FIG. 21, and other figures showing subsequent stages/states of prosthetic heart valves as described herein, the leaflet assembly 81 may form a shape of a tapered cylinder, cone, conical cylinder, or the like, wherein any of such shapes, or modified or imperfect/deformed versions thereof, can be referred to herein as ‘cylinder’ or ‘cone’ shaped assemblies/components. The interior of the leaflet assembly 81 may be considered the portions of the leaflets 42 radially within, and/or the space radially within, the cylinder/cone defined by the outer surface 206 of the leaflets 42 when assembled as shown in FIG. 21 (or as in FIGS. 17a, 22, 23, an others).

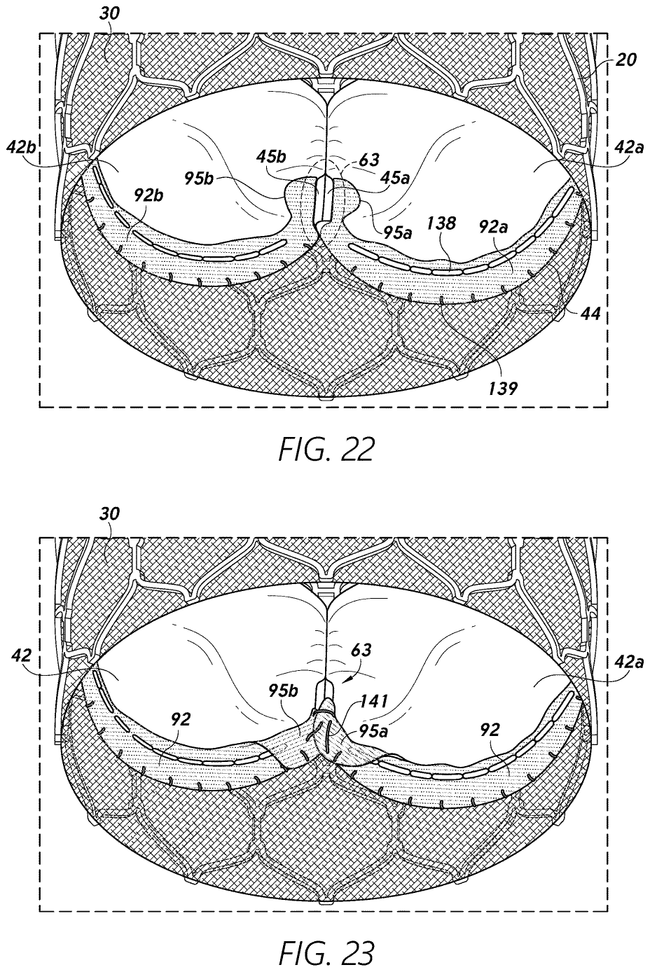

While FIG. 21 shows the leaflet assembly 81 prior to coupling to the valve frame, FIG. 22 shows the leaflet assembly 81 coupled to the valve frame 20, the leaflet assembly/structure 81 including the previously-executed radially-inward/inverted sub-commissure leaflet unions 63, with the joined leaflet edges 45a, 45b projecting into the inner diameter of the assembly 81, as shown. The reinforcement strips/skirts 92a, 92b associated with the cusp edges of the leaflets 42 can have terminal wing features 95a, 95b, which may be scallop-shaped or other flaring extension of the otherwise elongated and/or curved strip shape of the strips/skirts 92a, 92b. When the elongate portions of the strips/skirts 92 are sutured down/to along, the wing features 95 may be un-sutured/pinned, such that they form at least partially free flap features that can be folded down. Therefore, the features 95a, 95b can be considered ‘wing flaps.’ The cusp edges 44 of the leaflets can be sutured or otherwise attached to the main skirt 30 using, for example, a straight seam 138 and/or blanket stitch 139, which may run along the scallop line of the leaflet edges 44. The leaflet-to-leaflet coupling/union 63 can be implemented by a straight seam/stitch that is mostly/generally parallel with the internal, lateral edges 45a, 45b of the leaflets. Alternatively, as described above, the union 63 may be implemented as a comb stitch having a spine that is mostly/generally parallel with the edges 45a, 45b. While shown as bulb/bulge-shaped, it should be understood that the terminal wing/flap features 95a, 95b may have any suitable or desirable shape or form.

FIG. 23 shows a further step in the assembly process in which the reinforcement strip/skirt wings 95a, 95b are folded over at least a portion of the inverted leaflet edge union/stitch 63 and/or the cusp portions 44 of the leaflets and reinforcement strips/skirts 92. The folded-over material 95a, 95b can be tucked and/or flattened-down in a manner as to produce a generally-triangular overlapping form, as shown. In some implementations, one or more whip stitches 141 may be executed to hold the wings 95 down in the desired folded positions. While shown and described as terminal end portions of the respective lower/cusp edge strips/skirts 92, it should be understood that the wing/flap portions 95a, 95b may be associated with any lengthwise portion of the respective strips/skirts 92.

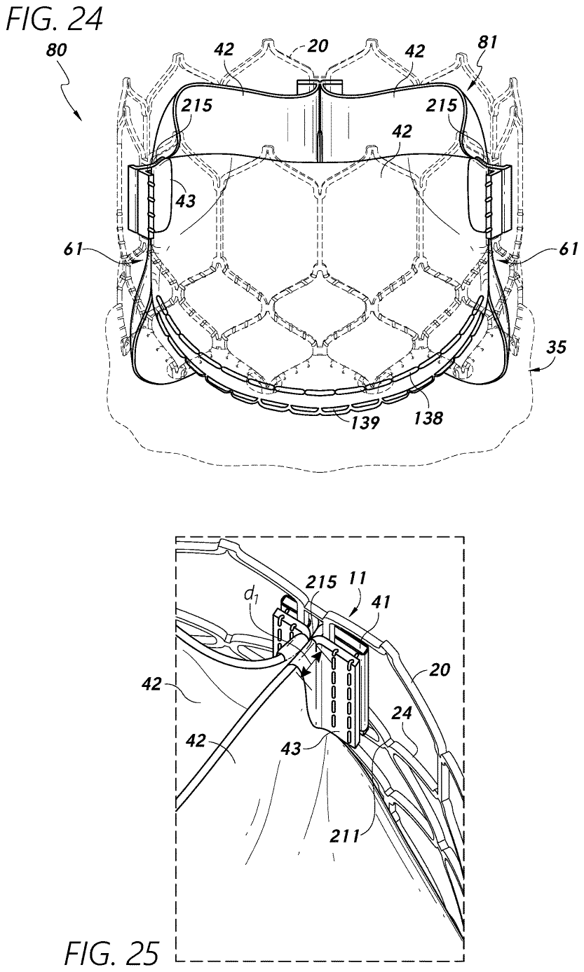

FIG. 24 shows an assembled prosthetic heart valve 80 having a leaflet assembly/structure 81 with an inverted sub-commissure leaflet union 61 for regulating blood flow in a low-pressure gradient hemodynamic situation in accordance with one or more examples. The main skirt of the device 80 is not shown for visual clarity, though an outer skirt 35 is illustrated. FIG. 25 is a close-up perspective view of a commissure portion 11 of a leaflet assembly/structure attached including radially-inward/inverted sub-commissure unions in accordance with one or more examples.

The leaflet assembly 81 includes commissure tabs 11 including a folded-down upper tab 43 folded at a fold portion 215, which may be the upper-most portion of the leaflets in the commissure areas. The folds 215 may have a radial dimension d₁ that has a bearing on the opening behavior of the leaflets 42. For example, the dimension d₁ may be about 2.5 mm or less, such as about 1 mm or less, which may provide desirable opening behavior for the leaflet assembly 81. With the illustrated dimensions and design, including the inverted leaflet union 61, the leaflets 81 may advantageously have an at least partially open configuration in low-or no-pressure gradient conditions.

In the illustrated example, the outer surface of the leaflets 42 may come into proximity with the inside surface 211 of the frame 20 and/or skirt (not shown for visual clarity; may be disposed inside the frame inner diameter, e.g., up to the boundary of the row of struts 24), such that no dead zones/gaps are present between the leaflets 42 and the frame/skirt. Such a configuration can minimize stasis by allowing for substantially all blood to be expelled as the heart cycles. The valve 80 can advantageously operate in a manner that eliminates or reduces static blood conditions that can be caused by frozen/lazy leaflets by promoting or ensuring regular opening of the prosthesis leaflets 81. Such implementations can be particularly helpful for implantation in the mitral valve position, where the flow conditions are generally lower than, for example, the aortic valve, such that there is less of an opportunity for leaflet washout.

### ADDITIONAL DESCRIPTION OF EXAMPLES

Provided below is a list of examples, each of which may include aspects of any of the other examples disclosed herein. Furthermore, aspects of any example described above may be implemented in any of the numbered examples provided below.

Example 1: A prosthetic heart valve comprising an annular frame, a skirt attached to the frame, and a leaflet structure disposed within the frame and coupled to a commissure portion of the frame, the leaflet structure including a first leaflet comprising a first lateral edge, and a second leaflet comprising a second lateral edge coupled to the first lateral edge of the first leaflet in a leaflet-to-leaflet union such that the first lateral edge and the second lateral edge are pointed radially inward with respect to an axis of the annular frame.

Example 2: The prosthetic heart valve of any example herein, in particular example 1, wherein the leaflet structure is coupled to the commissure portion of the frame above an upper edge of the skirt.

Example 3: The prosthetic heart valve of any example herein, in particular example 2, wherein the leaflet-to-leaflet union is at least partially radially covered by the skirt.

Example 4: The prosthetic heart valve of any example herein, in particular example 1, wherein the first leaflet comprises a first tab, the second leaflet comprises a second tab, and the first and second tabs pass through an open commissure cell of the commissure portion of the frame.

Example 5: The prosthetic heart valve of any example herein, in particular example 4, wherein the first lateral edge and the second lateral edge are positioned below the first tab and the second tab, respectively.

Example 6: The prosthetic heart valve of any example herein, in particular example 5, wherein the first and second tabs are connected by a flexible connector sheet sutured to both of the first and second tabs.

Example 7: The prosthetic heart valve of any example herein, in particular example 1, wherein the first and second lateral edges are part of a sub-commissure portion of the leaflet structure.

Example 8: The prosthetic heart valve of any example herein, in particular example 1, wherein the first lateral edge and the second lateral edge are directly sutured to one another by a straight seam that is parallel with the first and second lateral edges.

Example 9: The prosthetic heart valve of any example herein, in particular example 1, wherein the first lateral edge and the second lateral edge are directly sutured to one another by a comb stitch comprising a spine stitch and a plurality of tooth stitches.

Example 10: The prosthetic heart valve of any example herein, in particular example 1, further comprising a first reinforcing strip associated with a curved edge of the first leaflet, the first reinforcing strip including a first wing end portion, and a second reinforcing strip associated with a curved edge of the second leaflet, the second reinforcing strip including a second wing end portion, wherein the first wing end portion and the second wing end portion are folded over at least a portion of the leaflet-to-leaflet union.

Example 11: The prosthetic heart valve of any example herein, in particular example 10, further comprising one or more whip stitches securing the first and second wing end portions over the leaflet-to-leaflet union.

Example 12: The prosthetic heart valve of any example herein, in particular example 1, wherein an outer surface of at least a portion of the leaflet-to-leaflet union contacts an inner diameter of at least one of the frame or the skirt.

Example 13: The prosthetic heart valve of any example herein, in particular example 1, wherein the first leaflet and the second leaflet overlap in an area below the leaflet-to-leaflet union.

Example 14: The prosthetic heart valve of any example herein, in particular example 1, wherein curved bottom edges of the first and second leaflets are sutured to the skirt.

Example 15: A valve leaflet assembly comprising a first leaflet comprising a first curved lower edge, one or more first commissure tabs, and a first sub-commissure lateral edge positioned between the first curved lower edge and the one or more first commissure tabs. The valve leaflet assembly further comprises a second leaflet comprising a second curved lower edge, one or more second commissure tabs, and a second sub-commissure lateral edge positioned between the second curved lower edge and the one or more second commissure tabs, the second sub-commissure lateral edge being coupled to the first sub-commissure lateral edge of the first leaflet along a vertical seam such that the first and second sub-commissure lateral edges are positioned within an interior of the valve leaflet assembly.

Example 16: The valve leaflet assembly of any example herein, in particular example 15, wherein the at least one of the one or more first commissure tabs is coupled to at least one of the one or more second commissure tabs via a connector sheet coupled to an outer surface of the first and second leaflets.

Example 17: The valve leaflet assembly of any example herein, in particular example 15, wherein the vertical seam is formed by one or more stitches disposed within the interior of the valve leaflet assembly.

Example 18: The valve leaflet assembly of any example herein, in particular example 15, wherein an exterior portion of the vertical seam is at least partially radially overlapped outside by a sealing skirt coupled to a prosthetic heart valve frame.

Example 19: The valve leaflet assembly of any example herein, in particular example 18, wherein the exterior portion of the vertical seam contacts an inner surface of at least one of the sealing skirt or the prosthetic heart valve frame.

Example 20: The valve leaflet assembly of any example herein, in particular example 15, wherein the one or more first commissure tabs comprises an upper tab and a lower tab configured to be coupled to one another around a vertical commissure strut of a prosthetic heart valve frame.

Example 21: The valve leaflet assembly of any example herein, in particular example 15, wherein the vertical seam is formed by a straight stitch that is parallel with the first and second sub-commissure lateral edges.

Example 22: The valve leaflet assembly of any example herein, in particular example 15, wherein the vertical seam is formed by a comb stitch comprising a vertical spine stitch and a plurality of lateral tooth stitches.

Example 23: The valve leaflet assembly of any example herein, in particular example 15, further comprising a first reinforcing strip coupled to an inner surface of the first curved lower edge of the first leaflet, the first reinforcing strip including a first terminal wing portion, and a second reinforcing strip coupled to an inner surface of the second curved lower edge of the second leaflet, the second reinforcing strip including a second terminal wing portion, wherein the first terminal wing portion and the second terminal wing portion are folded over at least a portion of the first and second sub-commissure lateral edges within the interior of the valve leaflet assembly.

Example 24: The valve leaflet assembly of any example herein, in particular example 23, wherein the folded first and second terminal wing portions are stitched down onto at least one of the first sub-commissure lateral edge, the second sub-commissure lateral edge, the first reinforcing strip, or the second reinforcing strip.

Example 25: The valve leaflet assembly of any example herein, in particular example 15, wherein the first leaflet and the second leaflet radially and circumferentially overlap in an area below the vertical seam.

Example 26: The valve leaflet assembly of any example herein, in particular example 15, wherein first and second curved lower edges are sutured to a sealing skirt of a prosthetic heart valve.

Example 27: A method of coupling leaflets of a valve leaflet assembly, the method comprising providing a first leaflet including a first commissure tab, a first sub-commissure side, and a first curved lower cusp, providing a second leaflet including a second commissure tab, a second sub-commissure side, and a second curved lower cusp, and forming a plurality of leaflets into a cylinder, the plurality of leaflets including the first and second leaflets, at least in part by forming a leaflet-to leaflet union by coupling the first sub-commissure side to the second sub-commissure side to one another such that edges of the first and second sub-commissure sides point radially inwardly within the cylinder.

Example 28: The method of any example herein, in particular example 27, wherein said coupling the first sub-commissure side to the second sub-commissure side involves placing an outer surface of the first sub-commissure side to an outer surface of the second sub-commissure side, and suturing the outer surface of the first sub-commissure side to the outer surface of the second sub-commissure side.

Example 29: The method of any example herein, in particular example 28, further comprising deflecting edges of the first and second sub-commissure sides toward inner sides of the first and second sub-commissure sides.

Example 30: The method of any example herein, in particular example 28, wherein said suturing the outer surface of the first sub-commissure side to the outer surface of the second sub-commissure side involves executing a straight stitch through the first and second sub-commissure sides along edges of the first and second sub-commissure sides.

Example 31: The method of any example herein, in particular example 28, wherein said suturing the outer surface of the first sub-commissure side to the outer surface of the second sub-commissure side involves executing a spine stitch along edges of the first and second sub-commissure sides, and executing teeth stitches over the edges of the first and second sub-commissure sides perpendicular to the spine stitch.

Example 32: The method of any example herein, in particular example 27, further comprising coupling the first commissure tab to the second commissure tab via a connector strip.

Example 33: The method of any example herein, in particular example 27, further comprising suturing the first curved lower cusp and the second curved lower cusp to a sealing skirt of a heart valve.

Example 34: The method of any example herein, in particular example 33, further comprising folding wing flaps of cusp strips associated with the first leaflet and the second leaflet, respectively, over edges of the first and second sub-commissure sides.

Example 35: The method of any example herein, in particular example 34, further comprising suturing the wing flaps to the cusp strips.

Example 36. The method of any example herein, in particular example 27, further comprising attaching the first and second commissure tabs to one or more struts of an annular frame of a heart valve.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A prosthetic heart valve comprising:
an annular frame;
a skirt attached to the frame; and
a leaflet structure disposed within the frame and coupled to a commissure portion of the frame, the leaflet structure including:
a first leaflet comprising a first lateral edge; and
a second leaflet comprising a second lateral edge coupled to the first lateral edge of the first leaflet in a leaflet-to-leaflet union such that the first lateral edge and the second lateral edge are pointed radially inward with respect to an axis of the annular frame.

2. The prosthetic heart valve of claim 1, wherein:
the leaflet structure is coupled to the commissure portion of the frame above an upper edge of the skirt; and
the leaflet-to-leaflet union is at least partially radially covered by the skirt.

3. The prosthetic heart valve of claim 1, wherein:
the first leaflet comprises a first tab;
the second leaflet comprises a second tab; and
the first and second tabs pass through an open commissure cell of the commissure portion of the frame.

4. The prosthetic heart valve of claim 3, wherein:
the first lateral edge and the second lateral edge are positioned below the first tab and the second tab, respectively; and
the first and second tabs are connected by a flexible connector sheet sutured to both of the first and second tabs.

5. The prosthetic heart valve of claim 1, wherein the first and second lateral edges are part of a sub-commissure portion of the leaflet structure.

6. The prosthetic heart valve of claim 1, wherein the first lateral edge and the second lateral edge are directly sutured to one another by a straight seam that is parallel with the first and second lateral edges.

7. The prosthetic heart valve of claim 1, wherein the first lateral edge and the second lateral edge are directly sutured to one another by a comb stitch comprising a spine stitch and a plurality of tooth stitches.

8. The prosthetic heart valve of claim 1, further comprising:
a first reinforcing strip associated with a curved edge of the first leaflet, the first reinforcing strip including a first wing end portion; and
a second reinforcing strip associated with a curved edge of the second leaflet, the second reinforcing strip including a second wing end portion;
wherein the first wing end portion and the second wing end portion are folded over at least a portion of the leaflet-to-leaflet union.

9. The prosthetic heart valve of claim 8, further comprising one or more whip stitches securing the first and second wing end portions over the leaflet-to-leaflet union.

10. The prosthetic heart valve of claim 1, wherein:
an outer surface of at least a portion of the leaflet-to-leaflet union contacts an inner diameter of at least one of the frame or the skirt;
the first leaflet and the second leaflet overlap in an area below the leaflet-to-leaflet union; and
curved bottom edges of the first and second leaflets are sutured to the skirt.

11. A prosthetic heart valve leaflet assembly comprising:
a first leaflet comprising:
a first curved lower edge;
one or more first commissure tabs; and
a first sub-commissure lateral edge positioned between the first curved lower edge and the one or more first commissure tabs; and
a second leaflet comprising:
a second curved lower edge;
one or more second commissure tabs; and
a second sub-commissure lateral edge positioned between the second curved lower edge and the one or more second commissure tabs, the second sub-commissure lateral edge being coupled to the first sub-commissure lateral edge of the first leaflet along a vertical seam such that the first and second sub-commissure lateral edges are positioned within an interior of the valve leaflet assembly.

12. The prosthetic heart valve leaflet assembly of claim 11, wherein:
the at least one of the one or more first commissure tabs is coupled to at least one of the one or more second commissure tabs via a connector sheet coupled to an outer surface of the first and second leaflets; and
the vertical seam is formed by one or more stitches disposed within the interior of the valve leaflet assembly.

13. The prosthetic heart valve leaflet assembly of claim 11, wherein:
an exterior portion of the vertical seam is at least partially radially overlapped outside by a sealing skirt coupled to a prosthetic heart valve frame; and
the exterior portion of the vertical seam contacts an inner surface of at least one of the sealing skirt or the prosthetic heart valve frame.

14. The prosthetic heart valve leaflet assembly of claim 11, wherein:
the one or more first commissure tabs comprises an upper tab and a lower tab configured to be coupled to one another around a vertical commissure strut of a prosthetic heart valve frame;
the vertical seam is formed by a straight stitch that is parallel with the first and second sub-commissure lateral edges; and
the vertical seam is formed by a comb stitch comprising a vertical spine stitch and a plurality of lateral tooth stitches.

15. The prosthetic heart valve leaflet assembly of claim 11, further comprising:
a first reinforcing strip coupled to an inner surface of the first curved lower edge of the first leaflet, the first reinforcing strip including a first terminal wing portion;
a second reinforcing strip coupled to an inner surface of the second curved lower edge of the second leaflet, the second reinforcing strip including a second terminal wing portion; and
the first terminal wing portion and the second terminal wing portion are folded over at least a portion of the first and second sub-commissure lateral edges within the interior of the valve leaflet assembly.

16. The prosthetic heart valve leaflet assembly of claim 15, wherein the folded first and second terminal wing portions are stitched down onto at least one of the first sub-commissure lateral edge, the second sub-commissure lateral edge, the first reinforcing strip, or the second reinforcing strip.

17. The prosthetic heart valve leaflet assembly of claim 11, wherein:
the first leaflet and the second leaflet radially and circumferentially overlap in an area below the vertical seam; and
first and second curved lower edges are sutured to a sealing skirt of a prosthetic heart valve.

18. A method of coupling leaflets of a valve leaflet assembly, the method comprising:
providing a first leaflet including a first commissure tab, a first sub-commissure side, and a first curved lower cusp;
providing a second leaflet including a second commissure tab, a second sub-commissure side, and a second curved lower cusp; and
forming a plurality of leaflets into a cylinder, the plurality of leaflets including the first and second leaflets, at least in part by:
forming a leaflet-to leaflet union by coupling the first sub-commissure side to the second sub-commissure side to one another such that edges of the first and second sub-commissure sides point radially inwardly within the cylinder.

19. The method of claim 18, wherein said coupling the first sub-commissure side to the second sub-commissure side involves:
placing an outer surface of the first sub-commissure side to an outer surface of the second sub-commissure side; and
suturing the outer surface of the first sub-commissure side to the outer surface of the second sub-commissure side.

20. The method of claim 19, further comprising deflecting edges of the first and second sub-commissure sides toward inner sides of the first and second sub-commissure sides, wherein:
said suturing the outer surface of the first sub-commissure side to the outer surface of the second sub-commissure side involves executing a straight stitch through the first and second sub-commissure sides along edges of the first and second sub-commissure sides; and
said suturing the outer surface of the first sub-commissure side to the outer surface of the second sub-commissure side involves:
executing a spine stitch along edges of the first and second sub-commissure sides; and
executing teeth stitches over the edges of the first and second sub-commissure sides perpendicular to the spine stitch.
